(19) United States
(12) Patent Application Publication
(10) Pub. No.: US 2003/0175410 A1
(43) Pub. Date: Sep. 18, 2003

(54) Method and apparatus for preparing biomimetic scaffold

(76) Inventors: Phil G. Campbell, Pittsburgh, PA (US); Lee E. Weiss, Pittsburgh, PA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/391,458

(22) Filed: Mar. 18, 2003

Related U.S. Application Data
(60) Provisional application No. 60/365,451, filed on Mar. 18, 2002.

(51) Int. Cl. A61F 2/02

(52) U.S. Cl. 427/2.24; 435/396; 623/23.72; 118/664

(57) ABSTRACT

Methods, compositions, and apparatus for preparing biomimetic scaffolds are provided. The methods, compositions, and apparatus are compatible with both in situ and external scaffold preparation. Also provided are methods for preparing scaffolds having 3-D spatial and/or temporal gradients of therapeutic compounds, such as, growth factors, antibiotics, immunosuppressants, analgesics, etc.

### CROSS REFERENCE TO RELATED APPLICATIONS

This application claims the benefit of Provisional Patent Application No. 60/365,451 filed Mar. 18, 2002 which is hereby incorporated by reference in its entirety.

### BACKGROUND

Clinical use of grafts of living tissue have recently moved from direct implantation of freshly harvested fully formed tissue, e.g. skin grafts or organ transplants, to strategies involving seeding of cells and signaling molecules on matrices which will regenerate or encourage the regeneration of local structures. For certain tissues it may be desirable to provide mechanical support of the existing structure by replacement or substitution of the tissue for at least some of the healing period. Thus, a device or scaffold having a specific architecture may be used to encourage the migration, residence and proliferation of specific cell types as well as provide mechanical and structural support during healing.

In order to encourage cellular attachment and growth, the overall porosity of the device is important. Additionally, the individual pore diameter or size is an important factor in determining the ability of cells to migrate into, colonize, and differentiate while in the device (Martin, R B et al. Biomaterials, 14: 341, 1993). For skeletal tissues, bone and cartilage, guided support to reproduce the correct geometry and shape of the tissue is thought to be important. It is generally agreed that pore sizes of above 150 μm and preferably larger (Hulbert, et al., 1970; Klawitter, J. J, 1970; Piecuch, 1982; and Dennis, et al., 1992) and porosity greater than 50% are necessary for cell invasion of the carrier by bone forming cells. It has been further accepted that a tissue regenerating scaffold must be highly porous, greater than 50% and more preferably more than 90%, in order to facilitate cartilage formation.

It has been further recognized that not only the morphology of such devices but the materials of which they are composed will contribute to the regeneration processes as well as the mechanical strength of the device. For example, some materials are osteogenic and stimulate the growth of bone forming cells; some materials are osteoconductive, encouraging bone-forming cell migration and incorporation; and some are osteoinductive, inducing the differentiation of mesenchymal stem cells into osteoblasts. Materials which have been found to be osteogenic usually contain a natural or synthetic source of calcium phosphate. Osteoinductive materials include molecules derived from members of the transforming growth factor-beta (TGF-beta) gene superfamily including: bone morphogenetic proteins (BMPs) and insulin-like growth factors (IGFs).

It is well documented that the physiological processes of wound healing and tissue regeneration proceed sequentially with multiple cell types and that cellular factors play a role. For example, thrombi are formed and removed by blood elements, which are components of cascades regulating both coagulation and clot lysis. Fibroblasts, migrate into the thrombus and lay down collagen fibers. Angiogenic cells are recruited by chemotactic factors, derived from circulating precursors or released from cells, to form vascular tissue. Finally, various precursor cells differentiate to form specialized tissue. The concept of adding exogenous natural or synthetic factors in order to hasten the healing process is an area of intense exploration, and numerous growth factors, such as cytokines, angiogenic factors, and transforming factors, have been isolated, purified, sequenced, and cloned.

A variety of techniques such as fiber bonding, solvent-casting and particulate leaching, melt molding, three-dimensional (3-D) printing and stereo-lithography are currently employed for manufacturing scaffolds. However, a need still exists for methods or apparatus that permit improved formation of a scaffold or device having a 3-D spatial and/or concentration gradient of therapeutic or structural elements. The development of such techniques would greatly increase the effectiveness and clinical applicability of tissue engineering scaffolds. Scaffolds containing such gradients would provide a high level of control over the integration of an engineered tissue into a desired location in a patient.

It is therefore an object of the present invention to overcome these shortcomings in existing tissue engineering techniques, by providing a methods, compositions, and apparatus for the preparation of biomimetic scaffolds having 3-D gradients of structural and/or therapeutic elements. The methods, compositions, and apparatus of the invention are compatible with ex vivo and in situ tissue engineering.

### SUMMARY

The present disclosure provides methods and apparatuses for selectively depositing bio-ink solutions to build up a 3-D biomimetic scaffold structure. In one aspect, the disclosure provides a method for preparing such biomimetic scaffolds by co-depositing one or more of the bio-ink solutions. In another aspect, the disclosure provides methods for depositing the bio-ink solutions to provide a patterned 3-D concentration gradient of the bio-inks. In certain embodiments, the biomimetic scaffold structure has a spatial and temporal concentration gradient of the bio-ink solutions.

In one embodiment, bio-ink solutions are provided that are used to create the biomimetic scaffold structures. The bio-inks may be biocompatible in nature. The bio-inks may optionally be biodegradable and or bioresorbable. In general, the bio-inks may be characterized as structural, functional and/or therapeutic bio-inks. Structural bio-inks provide among other properties, mechanical properties, porosity, and increased surface area. Examples of such structural bio-inks include, without being limited to, hydrogel solutions, fibrinogen, thrombin, chitosan, collagen, alginate, poly(N-isopropylacrylamide), hyaluronate, polylactic acid (PLA), polyglycolic acid (PGA), and PLA-PGA co-polymers. In one exemplary embodiment, fibrinogen and thrombin are co-deposited to provide a fibrin matrix. In yet another embodiment, the fibrinogen may be cross-linked to growth factors.

Functional bio-inks may modify, preserve, or enhance a particular property. For example, among other properties, functional bio-inks may provide cell-adhesion properties, modulate cross-linking within the biomimetic scaffold structure, modulate the ionic concentration, and modulate the pH of the biomimetic scaffold structure. The cross-linking agent may be any biocompatible agent, such as naturally occurring or synthetic cross-linker, such as for example transglutaminase.

Therapeutic bio-inks may function in a number of ways to produce a biological effect in vivo, such as for example, to modulate the immune response, to promote wound healing, promote cell proliferation, promote cell differentiation, promote angiogenesis, vessel permeabilization. Examples of therapeutic bio-inks include, without limitation, agents that elicit a cellular response, including growth factors, cytokines, and hormones. Other examples of therapeutic bio-inks include, without limitation, neurotrophic factors, small molecules, signaling molecules, antibodies, antibiotics, analgesics, anti-toxins, nucleic acids, and tissue precursor cells.

In one embodiment, the bio-ink solidifies, or polymerizes or gels upon deposition. Such solidification, polymerization, or gelation may be due to a change in the micro-environment, such as, for example, a change in the temperature, pH, light, and/or ionic strength, or upon contact with another bio-ink. For example, a bio-ink may solidify, or polymerize or gel, at body-temperature.

In one embodiment, the biomimetic scaffold structure is prepared using a solid freeform fabrication system, such as, for example, an apparatus employing one or more focused micro-dispensing devices, which permits the co-depositing of bio-inks in a controllable manner. In certain embodiments, the bio-inks may be co-deposited in situ.

The biomimetic scaffolds disclosed herein are preferably biocompatible. The biomimetic scaffold may optionally be bioresorbable and/or biodegradable. In one embodiment, the biomimetic scaffold structure is implantable. The scaffold implant may be permanent or may be biodegradable. In another embodiment, a biomimetic scaffold may comprise a 3-D matrix wherein the scaffold has a patterned 3-D concentration gradient of therapeutic bio-inks.

In one embodiment, an apparatus for dispensing bio-inks onto a surface comprises a first micro-dispensing device fluidly connected to a source of a first bio-ink and configured to dispense a volume of the first bio-ink and a second micro-dispensing device fluidly connected to a source of a second bio-ink and configured to dispense a volume of the second bio-ink. The apparatus may also include a movable stage supporting the first micro-dispensing device and the second micro-dispensing device. The movable stage may be configured to move the first micro-dispensing device and the second dispensing device relative to the surface. During operation, the first micro-dispensing device and the second micro-dispensing device may be displaced by the stage relative to the surface and may selectively dispense a volume of the first bio-ink and a volume of the second bio-ink at a plurality of dispensing locations on the surface.

The first micro-dispensing device and the second micro-dispensing device may be focused to a focal point such that a dispensed volume of the first bio-ink converges with a dispensed volume of the second bio-ink at the focal point. During operation, the first micro-dispensing device and the second micro-dispensing device may selectively dispense a focused volume of the first bio-ink and second bio-ink at a plurality of dispensing locations on the surface.

The apparatus may include a third micro-dispensing device coupled to a source of a third bio-ink and configured to dispense a volume of the third bio-ink. The third micro-dispensing device may be supported by the movable stage and may be focused to the focal point of the first micro-dispensing device and the second micro-dispensing device such that a dispensed volume of the third bio-ink may converge with a dispensed volume of the first bio-ink and the second bio-ink at the focal point. The apparatus also may include additional micro-dispensing devices, each coupled to a source of bio-ink. For example, the apparatus may include a fourth micro-dispensing device coupled to a source of a fourth bio-ink and a fifth micro-dispensing device coupled to a source of a fifth bio-ink. Each of the additional micro-dispensing devices may be supported by the movable stage. Some or all of the micro-dispensing devices (e.g., the first, second, third, etc., micro-dispensing device) may be focused to a common focal point such that a dispensed volume of the bio-ink from two or more of the micro-dispensing device may converge at the common focal point.

The apparatus may include a control system coupled to the first micro-dispensing device and to the second micro-dispensing device. The control system may be configured to control the volume of first bio-ink and the volume of second bio-ink dispensed at each dispensing location on the surface.

Each micro-dispensing device may be an ink jet print head, a micro-dispensing solenoid valve, a syringe pump, or any other devices for dispensing small volumes of fluids. In certain exemplary embodiments, a suitable micro-dispensing device may dispense fluids in volumes of less than 100 nanoliters. In other exemplary embodiments, a suitable micro-dispensing device may dispense fluids in volumes of less than 100 picoliters.

Each micro-dispensing device may include a heating unit for heating the fluid being dispensed and/or may include a cooling unit for cooling the fluid being dispensed. Additionally, a heat source for heating at least some of the dispensing locations on the surface may be provided with apparatus. For example, the heat source may be an infrared heat source configured to direct infrared light onto at least some of the dispensing locations on the surface.

In accordance with another exemplary embodiment, an apparatus for in situ dispensing of a bio-ink on a subject may comprise a first micro-dispensing device fluidly connected to a source of a first bio-ink and configured to dispense a volume of the first bio-ink and a second micro-dispensing device fluidly connected to a source of a second bio-ink and configured to dispense a volume of the second bio-ink. The apparatus may include a movable stage supporting the first micro-dispensing device and the second micro-dispensing device. The movable stage may be configured to be connected to a subject and to move the first micro-dispensing device and the second micro-dispensing device relative to the subject. During operation, the first micro-dispensing device and the second micro-dispensing device may be displaced relative to the subject to selectively dispense a volume of the first bio-ink and a volume of the second bio-ink at a plurality of dispensing locations on the subject. The movable stage may be a stereotactic device or other device suitable for connecting medical instruments to a subject.

In accordance with a further exemplary embodiment, a hand-held instrument may comprise an instrument frame having a handle sized and shaped to be held by a user, a first micro-dispensing device coupled to the instrument frame and fluidly connected to a source of a of a first bio-ink, and a second micro-dispensing device coupled to the instrument frame and fluidly connected to a source of a second bio-ink. The first micro-dispensing device may be configured to dispense a volume of the first bio-ink and the second micro-dispensing device may be configured to dispense a volume of the second bio-ink.

### BRIEF DESCRIPTION OF THE DRAWINGS

These and other features and advantages of the apparatuses, methods, and compositions disclosed herein will be more fully understood by reference to the following detailed description in conjunction with the attached drawings in which like reference numerals refer to like elements through the different views. The drawings illustrate principles of the apparatuses, methods, and compositions disclosed herein and, although not to scale, show relative dimensions.

FIG. 1 is a schematic view of an exemplary embodiment of an apparatus for dispensing bio-inks onto a surface;

FIG. 2 is a schematic view of an exemplary embodiment of an apparatus for dispensing bio-inks onto a surface, illustrating a plurality of micro-dispensing devices;

FIG. 3 is a schematic view of an exemplary embodiment of an apparatus for in situ dispensing of a bio-ink on a subject, illustrating a plurality of micro-dispensing devices coupled to a stereotactic device;

FIG. 4 is a schematic view of an exemplary embodiment of a hand held apparatus for dispensing bio-inks;

FIG. 5 is a side elevational view in cross-section of an exemplary embodiment of an endoscopic instrument for dispensing bio-inks;

FIG. 6 is a diagram showing a fibrin biomimetic extracellular matrix (bECM) with spatial concentration gradients of FGF-2 and platelet derived growth factor (PDGF) and fibrin;

FIG. 7 is a diagram illustrating an in situ apparatus for dispensing bio-inks on a surface to form a biomimetic scaffold;

FIG. 8 is a photograph of an exemplary apparatus for co-dispensing bio-inks to form a biomimetic scaffold;

FIG. 9A is a photograph of a fibrin bECM;

FIG. 9B (top panel) is a non-illuminated photograph of a fibrin bECM (10 mm×10 mm) illustrating the gradient of a fluorescent tag, Cy3;

FIG. 9B (bottom panel) is a photograph of the fibrin bECM of FIG. 9B (top panel) with fluorescent imaging;

FIG. 9C is a photograph of a fibrin bECM, illustrating the gradient of fibrin porosity;

FIG. 10 is a schematic view showing an exemplary set of micro-dispensing devices for dispensing bio-inks, including fibrinogen (Fg), thrombin (Tr), tissue transglutaminase (TG), FGF-2, and a diluting buffer;

FIG. 11 is a schematic view of an apparatus for dispensing bio-inks on a surface to form a biomimetic scaffold such as a bECM;

FIGS. 12A-F are schematic views of exemplary biomimetic scaffold designs.

FIG. 13A is a schematic view of an apparatus for dispensing bio-inks, illustrating the dispensing of bio-inks onto the underside of polycarbonate membrane based culture plate to form a bECM;

FIG. 13B is a schematic view of the polycarbonate membrane based culture plate of FIG. 13A, illustrating the inversion of the bECM into the culture plate and cells plated in the insert well;

FIG. 14A is a photograph of a cutting device for cutting a hole in an egg as part of method of forming a biomimetic scaffold in an egg;

FIG. 14B is a photograph of an egg having a hole formed therein, illustrating a optically clear plastic insert positioned within the hole formed in the egg to facilitate viewing of a biomimetic scaffold positioned proximate the chorioallantoic membrane (CAM) of the egg;

FIG. 14B is a photograph of the egg of FIG. 14B, illustrating the CAM in situ;

FIG. 15A is a schematic view of an apparatus for dispensing bio-inks, illustrating the dispensing of bio-inks onto the underside of a Millicell tissue culture membrane insert to form a bECM;

FIG. 15B is a schematic view of the Millicell tissue culture membrane and bECM inverted onto the CAM of an egg;

FIG. 16A is a schematic view of a bECM design printed in situ in a calibration pattern in a critical-sized defect (CSD) in a rat cadaver;

FIG. 16B is a schematic view of a bECM design printed in situ in a CSD of a rat cadaver in the radial design illustrated in FIG. 12E;

FIG. 17A is a photograph of an empty CSD in the parietal bone of the rat clavarium; and

FIG. 17B is a photograph of in situ printing of fibrin with methylene blue into the CSD shown in FIG. 17A.

### DETAILED DESCRIPTION

General Description

To provide an overall understanding, certain illustrative embodiments will now be described; however, it will be understood by one of ordinary skill in the art that the systems, methods, and compositions described herein can be adapted and modified to provide systems, methods, and compositions for other suitable applications and that other additions and modifications can be made without departing from the scope of the present disclosure.

Unless otherwise specified, the illustrated embodiments can be understood as providing exemplary features of varying detail of certain embodiments, and therefore unless otherwise specified, features, components, modules, and/or aspects of the illustrations can be combined, separated, interchanged, and/or rearranged without departing from the disclosed systems or methods.

The present disclosure provides methods, compositions and apparatus for creating biomimetic structures. In accordance with the disclosure, solid freeform fabrication (SFF) processes and apparatus are used in a layering manufacturing process to build up shapes by incremental materials deposition and fusion of thin cross-sectional layers. In certain embodiments, the biomimetic structures are created ex vivo and then administered to a patient (e.g., surgically implanted or attached to a host organism). Alternatively, biomimetic structures may be manufactured in situ directly at a desired location (e.g., a wound, bone fracture, etc.).

In certain embodiments, the biomimetic structure may be fabricated out of biocompatible materials which are designed for long term or permanent implantation into a host organism. For example, a graft may be used to repair or replace damaged tissue or an artificial organ may be used to replace a diseased or damaged organ (e.g., liver, bone, heart, etc.). Alternatively, biomimetics may be fabricated out of biodegradable materials to form temporary structures. For example, a bone fracture may be temporarily sealed with a biodegradable biomimetic that will undergo controlled biodegradation occurring concomitantly with bioremodeling by the host's cells.

The 3-D structure of the biomimetic may be fabricated directly using SFF. For example, magnetic resonance imaging (MRI) or computerized axial tomography (CAT) scans may be used to determine the 3-D shape of an in vivo structure which is to be repaired or replaced. Computer-aided-design (CAD) or computer-aided-manufacturing (CAM) is then used to facilitate fabrication of the 3-D structure using SFF as described herein. Alternatively, the methods and apparatus disclosed herein may be used to produce a non-specific 3-D structure (e.g., a block or cube), which is then cut or molded into the desired shape (e.g., using a laser, saw, blade, etc.).

Additionally, the methods and apparatus disclosed herein may be used to create biomimetics with specific microstructural organization such that the biomimetic has the anatomical, biomechanical, and biochemical features of naturally occurring tissues, or engineering designs that are biologically inspired. The microstructural organization includes the spatial concentration of one or more bio-inks, the degree of porosity of the biomimetic, and/or channels that run through the 3-D structure for improved cell invasion, vascularization and nutrient diffusion.

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “biocompatible” refers to the absence of stimulation of a severe, long-lived or escalating biological response to an implant or coating, and is distinguished from a mild, transient inflammation which typically accompanies surgery or implantation of foreign objects into a living organism.

The terms “biodegradable” and “bioerodible” refer to the dissolution of an implant or coating into constituent parts that may be metabolized or excreted, under the conditions normally present in a living tissue. In exemplary embodiments, the rate and/or extent of biodegradation or bioerosion may be controlled in a predictable manner.

The term “bio-ink” is intended to include any material, whether liquid, solid or semisolid, that is suitable for deposition as part of the construction of a biomimetic scaffold. Any material that is biocompatible or biodegradable is suitable for use as a bio-ink in accordance with the present disclosure. Generally, bio-inks may be characterized as structural, functional or therapeutic. “Structural bio-inks” are capable of forming the 3-D scaffold of the biomimetic structure. Bio-inks which modify, preserve or enhance a characteristic (e.g., pH, porosity, surface adhesion, etc.) of the biomimetic scaffold are termed “functional bio-inks”. “Therapeutic bio-inks” are capable of producing a biological effect in vivo (e.g., stimulation of cell division, migration or apoptosis; stimulation or suppression of an immune response; anti-bacterial activity; etc.).

The term “biomimetic scaffold” includes essentially any assembly of materials that is designed to imitate a biological structure, such as, for example, by imitating an aspect of fine structure (e.g. pore size and/or abundance) or by imitating the ability to support adhesion and/or growth of at least one appropriate cell type.

The term “co-depositing” describes the placement of two or more substances, usually bio-inks, at the same position in, for example, a biomimetic scaffold. Substances may be co-deposited simultaneously or non-simultaneously (for example, sequentially).

A “concentration gradient” is one or more dimensions (whether in space or time) along which the concentration and/or accessibility of one or more substances may vary. The term is intended to include gradients in which the concentration is uniform throughout (i.e. a flat line gradient) as well as gradients in which the concentration varies. Concentration gradients include both linear gradients (i.e., gradients which increase or decrease at a continuous rate) and non-linear gradients. A “spatial concentration gradient” is a concentration gradient in which the concentration may vary along one or more spatial dimensions. A “temporal concentration gradient” is a concentration gradient in which the concentration may vary over time. In certain embodiments, a temporal concentration gradient may be created by capsules designed for timed release of one or more substances. In other embodiments, a temporal concentration gradient may be created through spatial patterning or structural design of the scaffold. For example, a temporal concentration gradient may be created by immobilizing (e.g., via absorption or chemical crosslinking either directly or via an intermediate) one or more substances on the scaffold in a pattern. In this manner, the timing of interaction with the substances will be controlled based on the time it takes for a cell to come into direct contact with the substances immobilized on the scaffold. In another example, a temporal concentration gradient may be created in a biomimetic scaffold having a fixed porosity by including one or more substances at a remote location on or within the scaffold. In this manner, interaction with the substances will be delayed during the period of time that it takes a cell to invade the scaffold and reach the remote location within the scaffold. Alternatively, a temporal gradient may be created in a scaffold using a variable porosity to control the rate of cell invasion into the scaffold. As cells encounter a higher porosity environment, the rate of invasion will be slowed, thus delaying interaction with one or more substances located in an area having a higher porosity. In still another embodiment, a temporal gradient may be created using biodegradable or bioresorbable scaffold. As the scaffold breaks down over time, the porosity of the scaffold may decrease thus permitting cell invasion at a more rapid rate. Alternatively, break down of the scaffold may expose a previously inaccessible area within the scaffold. A “3-D concentration gradient” is a set of three orthogonal spatial dimensions in which the concentration of one or more substances may vary independently along each dimension.

“Cross-linking” is the formation of a covalent attachment between two entities, typically polymer subunits that are not otherwise attached at that point.

The term “gelation” refers to the phase transition that a polymer undergoes when it increases in viscosity and transforms from a fluid state into a semi-solid material, or gel. At this transition point, the molecular weight (weight average) of the polymer matrix becomes “infinite” due to the formation of an essentially continuous matrix throughout the nascent gel. Polymerization can continue beyond the point of gelation through the incorporation of additional polymer units into the gel matrix. As used herein, “gel” may include both the semisolid gel state and the high viscosity state that exists above the gelation temperature.

The term “Gelation temperature” refers to the temperature at which a polymer undergoes reverse thermal gelation, i.e. the temperature below which the polymer is soluble in water and above which the polymer undergoes phase transition to increase in viscosity or to form a semi-solid gel. Because gelation does not involve any change in the chemical composition of the polymer, the gel may spontaneously reverse to the lower viscosity fluid form when cooled below the gelation temperature. The gelation temperature may also be referred to as the gel-solution (or gel-sol) transition temperature.

A “hydrogel” is defined as a substance formed when a polymer (natural or synthetic) becomes a 3-D open-lattice structure that entraps solution molecules, typically water, to form a gel. A polymer may form a hydrogel by, for example, aggregation, coagulation, hydrophobic interactions, cross-linking, salt bridges, etc. Where a hydrogel is to be used as part of a scaffold onto which cells will be seeded, the hydrogel should be non-toxic to the cells.

A “hydrogel solution” is a solute and a solvent comprising a substance that if subjected to the appropriate conditions, such as temperature, salt concentration, pH, the presence of a protease, the presence of a binding partner, etc., becomes a hydrogel or part of a hydrogel. The term “solution” in a hydrogel solution is intended to include true solutions as well as suspensions, such as colloidal suspensions, and other fluid materials where one component is not truly solubilized.

A “mechanical property” of a biomimetic scaffold includes essentially any property that provides some description for how the scaffold responds to the application of an external force. Exemplary mechanical properties include tensile strength, compressional strength, flexural strength, impact strength, elongation, modulus, toughness, etc.

The term “minimal-invasive surgery,” or “MIS,” refers to surgical procedures for treatment, diagnosis, and/or examination of one or more regions of a patient's body using surgical and diagnostic instruments specially developed to reduce the amount of physical trauma associated with the procedure. Generally, MIS involves instruments that may be passed through natural or surgically created openings of small diameter into a body to their location of use so that examinations and minor surgical interventions are possible with substantially less stress being imposed on the patient, for example, without general anesthesia. MIS may be accomplished using visualization methods such as fiberoptic or microscopic means. Examples of MIS include, for example, arthoscopic surgery, laparoscopic surgery, endoscopic surgery, thoracic surgery, neurosurgery, bladder surgery, gastrointestinal tract surgery, etc.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “polymerize” means to form an aggregate of multiple subunits, where the exact number of subunits in an aggregate is not precisely controlled by the properties of the aggregate itself. For example, “polymerize” does not refer to the formation of a hexameric enzyme complex that is designed to be consistently hexameric. However, the formation of hexamers of, for example, fibrin or actin, is a polymerization. Polymers are generally elongate, but may be of any shape, including a globular aggregate.

The term “polypeptide”, and the terms “protein” and “peptide” which are used interchangeably herein, refers to a polymer of amino acids.

A “subject” is essentially any organism, although usually a vertebrate, and most typically a mammal, such as a human or a non-human mammal.

The term “therapeutically effective amount” refers to that amount of a modulator, drug or other molecule that is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

As used herein, the term “tissue” refers to an aggregation of similarly specialized cells united in the performance of a particular function. Tissue is intended to encompass all types of biological tissue including both hard and soft tissue, including connective tissue (e.g., hard forms such as osseous tissue or bone) as well as other muscular or skeletal tissue.

The term “vector” refers to a nucleic acid capable of transporting another nucleic acid to which it has been linked. One type of vector which may be used herein is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules that, in their vector form are not bound to the chromosome. In the present specification, “plasmid” and “vector” are used interchangeably as the plasmid is the most commonly used form of vector. However, the present dislcosure is intended to include such other forms of expression vectors which serve equivalent functions and which become known in the art subsequently hereto.

Bio-Inks

The SFF methods and apparatus disclosed herein may use bio-inks to generate biomimetic structures with the aid of computer controlled micro-dispensing devices. Any material that is biocompatible or biodegradable is suitable for use as a bio-ink in accordance with the present disclosure. Generally, bio-inks may be characterized as structural, functional or therapeutic. Structural bio-inks are capable of forming the 3-D scaffold of the biomimetic structure. Bio-inks that modify, preserve or enhance a characteristic (e.g., pH, porosity, surface adhesion, etc.) of the biomimetic scaffold are characterized as functional. Therapeutic bio-inks are capable of producing a biological effect in vivo (e.g., stimulation of cell division, migration or apoptosis; stimulation or suppression of an immune response; anti-bacterial activity; anti-toxins; analgesics; etc.).

Structural Bio-inks

Structural bio-inks may comprise natural or synthetic organic polymers that can be gelled, or polymerized or solidified (e.g., by aggregation, coagulation, hydrophobic interactions, or cross-linking) into a 3-D open-lattice structure that entraps water or other molecules, e.g., to form a hydrogel. Structural bio-inks may comprise a single polymer or a mixture of two or more polymers in a single ink. Additionally, two or more structural bio-inks may be co-deposited so as to form a polymeric mixture at the site of deposition. Polymers used in bio-ink compositions may be biocompatible, biodegradable and/or bioerodible and may act as adhesive substrates for cells. In exemplary embodiments, structural bio-inks are easy to process into complex shapes and have a rigidity and mechanical strength suitable to maintain the desired shape under in vivo conditions.

In certain embodiments, the structural bio-inks may be non-resorbing or non-biodegradable polymers or materials. Such non-resorbing bio-inks may be used to fabricate materials which are designed for long term or permanent implantation into a host organism. In exemplary embodiments, non-biodegradable structural bio-inks may be biocompatible. Examples of biocompatible non-biodegradable polymers which are useful as bio-inks include, but are not limited to, polyethylenes, polyvinyl chlorides, polyamides such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates such as ethylene vinyl acetate copolymer, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art. Other biocompatible nondegradable polymers that are useful in accordance with the present disclosure include polymers comprising biocompatible metal ions or ionic coatings which can interact with DNA. Such metal ions include, but are not limited to gold and silver ions, Al³⁺, Fe³⁺, Fe²⁺, Mg²⁺, and Mn²⁺. In exemplary embodiments, gold and silver ions may be used, for example, for inhibiting inflammation, binding DNA, and inhibiting infection and thrombosis.

In other embodiments, the structural bio-inks may be a “bioerodible” or “biodegradable” polymer or material. Such bioerodible or biodegradable bio-inks may be used to fabricate temporary structures. In exemplary embodiments, biodegradable or bioerodible structural bio-inks may be biocompatible. Examples of biocompatible biodegradable polymers which are useful as bio-inks include, but are not limited to, polylactic acid, polyglycolic acid, polycaprolactone, and copolymers thereof, polyesters such as polyglycolides, polyanhydrides, polyacrylates, polyalkyl cyanoacrylates such as n-butyl cyanoacrylate and isopropyl cyanoacrylate, polyacrylamides, polyorthoesters, polyphosphazenes, polypeptides, polyurethanes, polystyrenes, polystyrene sulfonic acid, polystyrene carboxylic acid, polyalkylene oxides, alginates, agaroses, dextrins, dextrans, polyanhydrides, biopolymers such as collagens and elastin, alginates, chitosans, glycosaminoglycans, and mixtures of such polymers.

In still other embodiments, a mixture of non-biodegradable and bioerodible and/or biodegradable bio-inks may be used to form a biomimetic structure of which part is permanent and part is temporary.

In certain embodiments, the structural bio-ink composition is solidified or set upon exposure to a certain temperature; by interaction with ions, e.g., copper, calcium, aluminum, magnesium, strontium, barium, tin, and di-, tri- or tetra-functional organic cations, low molecular weight dicarboxylate ions, sulfate ions, and carbonate ions; upon a change in pH; or upon exposure to radiation, e.g., ultraviolet or visible light. In an exemplary embodiment, the structural bio-ink is set or solidified upon exposure to the body temperature of a mammal, e.g., a human being. The bio-ink composition can be further stabilized by cross-linking with a polyion.

In an exemplary embodiment, bio-inks may comprise naturally occurring substances, such as, fibrinogen, fibrin, thrombin, chitosan, collagen, alginate, poly(N-isopropylacrylamide), hyaluronate, albumin, collagen, synthetic polyamino acids, prolamines, polysaccharides such as alginate, heparin, and other naturally occurring biodegradable polymers of sugar units.

In certain embodiments, structural bio-inks may be ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix. In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Polyphosphazenes are polymers with backbones consisting of nitrogen and phosphorous atoms separated by alternating single and double bonds. Each phosphorous atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups.

Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl. Bioerodible or biodegradable polymers, i.e., polymers that dissolve or degrade within a period that is acceptable in the desired application (usually in vivo therapy), will degrade in less than about five years or in less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Methods for synthesis and the analysis of various types of polyphosphazenes are described in U.S. Pat. Nos. 4,440,921, 4,495,174, and 4,880,622. Methods for the synthesis of the other polymers described above are known to those skilled in the art. See, for example Concise Encyclopedia of Polymer Science and Polymeric Amines and Ammonium Salts, E. Goethals, editor (Pergamen Press, Elmsford, N.Y. 1980). Many polymers, such as poly(acrylic acid), alginates, and PLURONICS™, are commercially available.

Water soluble polymers with charged side groups are cross-linked by reacting the polymer with an aqueous solution containing multivalent ions of the opposite charge, either multivalent cations if the polymer has acidic side groups, or multivalent anions if the polymer has basic side groups. Cations for cross-linking the polymers with acidic side groups to form a hydrogel include divalent and trivalent cations such as copper, calcium, aluminum, magnesium, and strontium. Aqueous solutions of the salts of these cations are added to the polymers to form soft, highly swollen hydrogels and membranes.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions are added to the polymers to form soft, highly swollen hydrogels and membranes, as described with respect to cations.

Also, a variety of polycations can be used to complex and thereby stabilize the polymer hydrogel into a semi-permeable surface membrane. Examples of one polycation is poly-L-lysine. There are also natural polycations such as the polysaccharide, chitosan.

For purposes of preventing the passage of antibodies across the membrane but allowing passage of nutrients essential for cellular growth and metabolism, a useful macromer/polymer size is in the range of between 10,000 D and 30,000 D. Smaller macromers result in polymer matrices of a higher density with smaller pores.

In other embodiments, the structural bio-inks may be temperature-dependent or thermosensitive hydrogels. These hydrogels must have so-called “reverse gelation” properties, i.e., they are liquids at or below room temperature, and gel when warmed to higher temperatures, e.g., body temperature. Thus, these hydrogels can be easily applied at or below room temperature as a liquid and automatically form a semi-solid gel when warmed to body temperature. Examples of such temperature-dependent hydrogels are PLURONICS™ (BASF-Wyandotte), such as polyoxyethylene-polyoxypropylene F-108, F-68, and F-127, poly (N-isopropylacrylamide), and N-isopropylacrylamide copolymers.

These copolymers can be manipulated by standard techniques to affect their physical properties such as porosity, rate of degradation, transition temperature, and degree of rigidity. For example, the addition of low molecular weight saccharides in the presence and absence of salts affects the lower critical solution temperature (LCST) of typical thermosensitive polymers. In addition, when these gels are prepared at concentrations ranging between 5 and 25% (W/V) by dispersion at 4° C., the viscosity and the gel-sol (gel-solution) transition temperature are affected, the gel-sol transition temperature being inversely related to the concentration. These gels have diffusion characteristics capable of allowing cells to survive and be nourished.

U.S. Pat. No. 4,188,373 describes using PLURONIC™ polyols in aqueous compositions to provide thermal gelling aqueous systems. U.S. Pat. Nos. 4,474,751, 4,474,752, 4,474,753, and 4,478,822 describe drug delivery systems which utilize thermosetting polyoxyalkylene gels; with these systems, both the gel transition temperature and/or the rigidity of the gel can be modified by adjustment of the pH and/or the ionic strength, as well as by the concentration of the polymer.

In yet other embodiments, structural bio-inks may be pH-Dependent Hydrogels. These hydrogels are liquids at, below, or above specific pH values, and gel when exposed to specific pHs, e.g., 7.35 to 7.45, the normal pH range of extracellular fluids within the human body. Thus, these hydrogels can be easily applied in the body as a liquid and automatically form a semi-solid gel when exposed to body pH. Examples of such pH-dependent hydrogels are TETRONICS™ (BASF-Wyandotte) polyoxyethylene-polyoxypropylene polymers of ethylene diamine, poly(diethyl aminoethyl methacrylate-g-ethylene glycol), and poly(2-hydroxymethyl methacrylate). These copolymers can be manipulated by standard techniques to affect their physical properties.

In certain embodiments, structural bio-inks may be light solidified hydrogels, e.g., hydrogels that may be solidified by either visible or ultraviolet light. These hydrogels are made of macromers including a water soluble region, a biodegradable region, and at least two polymerizable regions as described in U.S. Pat. No. 5,410,016. For example, the hydrogel can begin with a biodegradable, polymerizable macromer including a core, an extension on each end of the core, and an end cap on each extension. The core is a hydrophilic polymer, the extensions are biodegradable polymers, and the end caps are oligomers capable of cross-linking the macromers upon exposure to visible or ultraviolet light, e.g., long wavelength ultraviolet light.

Examples of such light solidified hydrogels can include polyethylene oxide block copolymers, polyethylene glycol polylactic acid copolymers with acrylate end groups, and 10K polyethylene glycol-glycolide copolymer capped by an acrylate at both ends. As with the PLURONIC™ hydrogels, the copolymers comprising these hydrogels can be manipulated by standard techniques to modify their physical properties such as rate of degradation, differences in crystallinity, and degree of rigidity.

In other embodiments, structural bio-inks may be a “bioerodible” or “biodegradable” synthetic polymer. Suitable polymers include, for example, bioerodible polymers such as poly(lactide) (PLA), poly(glycolic acid) (PGA), poly(lactide-co-glycolide) (PLGA), poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, teflon™, and nylon. In an exemplary embodiment, the structural bio-ink comprises a PLA/PGA copolymer that is biodegradable.

The speed of erosion of a scaffold produced from a bioerodible or biodegradable structural bio-ink is related to the molecular weights of the polymer contained in the bio-ink. Higher molecular weight polymers (e.g., with average molecular weights of 90,000 or higher) produce biomimetic scaffolds which retain their structural integrity for longer periods of time, while lower molecular weight polymers (e.g., average molecular weights of 30,000 or less) produce biomimetic scaffolds which erode much more quickly.

Functional Bio-Inks

Functional bio-inks are capable of modifying, preserving or enhancing one or more characteristics of the biomimetic scaffold, including, ionic concentration; pH; speed and/or extent of cross-linking of a structural bio-ink; speed and/or extent of setting or solidification of a structural bio-ink; speed and/or extent of degradation; porosity; rigidity; surface adhesion properties; modification of bioavailability, residence time and/or mass transport of a therapeutic bio-ink; and other characteristics of the 3-D biomimetic structure.

In certain embodiments, suitable functional bio-inks for improving surface adhesion of the biomimetic scaffold include nonfibrillar collagen, fibrillar collagen, mixtures of nonfibrillar and fibrillar collagen, methyl alpha-cyanoacrylate, methacrylate, 2-cyano-2-propenoic acid methyl ester, methyl 2-cyanoacrylate, 2-cyanoacrylic acid methyl ester, an n-butyl cyanoacrylate based glue, fibronectins, ICAMs, E-cadherins, and antibodies that specifically bind a cell surface protein (for example, an integrin, ICAM, selectin, or E-cadherin), peptides containing “RGD” integrin binding sequence, or variations thereof known to affect cellular attachment, or other biologically active cell attachment mediators.

In other embodiments, the functional bio-ink is a poly-vinyl alcohol, gelatin, hyaluranate, or a poly ethylene glycol.

In certain embodiments, the functional bio-ink may be a component that either augments (including, for example, a protease) or retards (including, for example, a protease inhibitor which may be a protein, peptide, or chemical) degradation of the 3-D biomimetic scaffold.

In other embodiments, the functional bio-ink is a buffer for maintaining, stabilizing or modulating pH.

In still other embodiments, the functional bio-ink is tissue transglutaminase Factor XIII (Factor XIII or tTG).

Therapeutic Bio-Inks

Therapeutic bio-inks are capable of producing a biological effect in vivo (e.g., stimulation or suppression of cell division, migration or apoptosis; stimulation or suppression of an immune response; anti-bacterial activity; etc.). Therapeutic bio-inks may comprise one or more agents, as described more fully below, in a single ink.

In certain embodiments, therapeutic bio-inks may be substances that enhance or exclude particular varieties of cellular or tissue ingrowth. Such substances include, for example, osteoinductive, angiogenic, mitogenic, or similar substances, such as transforming growth factors (TGFs), for example, TGF-alpha, TGF-beta-1, TGF-beta-2, TGF-beta-3; fibroblast growth factors (FGFs), for example, acidic and basic fibroblast growth factors (aFGF and bFGF); platelet derived growth factors (PDGFs); platelet-derived endothelial cell growth factor (PD-ECGF); tumor necrosis factor alpha (TNF-alpha); tumor necrosis factor beta (TNF-b); epidermal growth factors (EGFs); connective tissue activated peptides (CTAPs); osteogenic factors, for example, for example, BMP-1, BMP-2, BMP-3MP-4, BMP-5, BMP-6, BMP-7, BMP-8, BMP-9; insulin-like growth factor (IGF), for example, IGF-I and IGF-II; erythropoietin; heparin binding growth factor (hbgf); vascular endothelium growth factor (VEGF); hepatocyte growth factor (HGF); colony stimulating factor (CSF); macrophage-CSF (M-CSF); granulocyte/macrophage CSF (GM-CSF); nitric oxide synthase (NOS); nerve growth factor (NGF); muscle morphogenic factor (MMP); Inhibins (for example, Inhibin A, Inhibin B); growth differentiating factors (for example, GDF-1);Activins (for example, Activin A, Activin B, Activin AB); angiogenin; angiotensin; angiopoietin; angiotropin; antiangiogenic antithrombin (aaAT); atrial natriuretic factor (ANF); betacellulin; endostatin; endothelial cell-derived growth factor (ECDGF); endothelial cell growth factor (ECGF); endothelial cell growth inhibitor; endothelial monocyte activating polypeptide (EMAP); endothelial cell-viability maintaining factor; endothelin (ET); endothelioma derived mobility factor (EDMF); heart derived inhibitor of vascular cell proliferation; hematopoietic growth factors; erythropoietin (Epo); interferon (IFN); interleukins (IL); oncostatin M; placental growth factor (PlGF); somatostatin; transferring; thrombospondin; vasoactive intestinal peptide; and biologically active analogs, fragments, and derivatives of such growth factors.

In exemplary embodiments, the therapeutic bio-inks are growth factors, angiogenic factors, compounds selectively inhibiting ingrowth of fibroblast tissue such as anti-inflammatories, and compounds selectively inhibiting growth and proliferation of transformed (cancerous) cells. These factors may be utilized to control the growth and function of cells contained within or surrounding the biomimetic scaffold, including, for example, the ingrowth of blood and/or the deposition and organization of fibrous tissue around the biomimetic scaffold.

In other embodiments, the therapeutic bio-inks may be other biologically active molecules that exert biological effects in vivo, including, for example, enzymes, receptors, receptor antagonists or agonists, hormones, growth factors, autogenous bone marrow, antibiotics, antimicrobial agents and antibodies.

In certain embodiments, therapeutic bio-inks may be pharmaceutical compositions or drugs, including small organic molecules, including, for example, antibiotics and anti-inflammatories.

In still other embodiments, the therapeutic bio-inks may be polynucleotides. Examples of polynucleotides which are useful as bio-inks include, but are not limited to, nucleic acids and fragments of nucleic acids, including, for example, DNA, RNA, cDNA and recombinant nucleic acids; naked DNA, cDNA, and RNA; genomic DNA, cDNA or RNA; oligonucleotides; aptomeric oligonucleotides; ribozymes; anti-sense oligonucleotides (including RNA or DNA); DNA coding for an anti-sense RNA; DNA coding for tRNA or rRNA molecules (i.e., to replace defective or deficient endogenous molecules); double stranded small interfering RNAs (siRNAs); polynucleotide peptide bonded oligos (PNAs); circular or linear RNA; circular single-stranded DNA; self-replicating RNAs; mRNA transcripts; catalytic RNAs, including, for example, hammerheads, hairpins, hepatitis delta virus, and group I introns which may specifically target and/or cleave specific RNA sequences in vivo; polynucleotides coding for therapeutic proteins or polypeptides, as further defined herein; chimeric nucleic acids, including, for example, DNA/DNA hybrids, RNA/RNA hybrids, DNA/RNA hybrids, DNA/peptide hybrids, and RNA/peptide hybrids; DNA compacting agents; and gene/vector systems (i.e., any vehicle that allows for the uptake and expression of nucleic acids), including nucleic acids in a non-infectious vector (i.e., a plasmid) and nucleic acids in a viral vector. In an exemplary embodiment, chimeric nucleic acids, include, for example, nucleic acids attached to a peptide targeting sequences that directs the location of the chimeric molecule to a location within a body, within a cell, or across a cellular membrane (i.e., a membrane translocating sequence (“MTS”)). In another embodiment, a nucleic acid may be fused to a constitutive housekeeping gene, or a fragment thereof, which is expressed in a wide variety of cell types.

In certain embodiments, polynucleotides delivered by non-viral methods may be formulated or associated with nanocaps (e.g., nanoparticulate CaPO₄), colloidal gold, nanoparticulate synthetic polymers, and/or liposomes. In an exemplary embodiment, polynucleotides may be associated with QDOT™ Probes (www.qdots.com).

In certain embodiments, polynucleotides useful as therapeutic bio-inks may be modified so as to increase resistance to nucleases, e.g. exonucleases and/or endonucleases, and therefore have increased stability in vivo. Exemplary modifications include, but are not limited to, phosphoramidate, phosphothioate and methylphosphonate analogs of nucleic acids (see also U.S. Pat. Nos. 5,176,996; 5,264,564; and 5,256,775).

In certain embodiments, the therapeutic bio-ink is a polynucleotide that is contained within a vector. Vectors suitable for use herein, include, viral vectors or vectors derived from viral sources, for example, adenoviral vectors, herpes simplex vectors, papilloma vectors, adeno-associated vectors, retroviral vectors, pseudorabies virus, alpha-herpes virus vectors, and the like. A thorough review of viral vectors, particularly viral vectors suitable for modifying nonreplicating cells, and how to use such vectors in conjunction with the expression of polynucleotides of interest can be found in the book Viral Vectors: Gene Therapy and Neuroscience Applications Ed. Caplitt and Loewy, Academic Press, San Diego (1995). In other embodiments, vectors may be non-infectious vectors, or plasmids. Suitable non-infectious vectors, include, but are not limited to, mammalian expression vectors that contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and.eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17.

In certain embodiments, the therapeutic bio-inks (i.e., polypeptides, polynucleotides, small molecules, drugs, cells, etc.) may be mixed with or encapsulated in a substance that facilitates its delivery to and/or uptake by a cell. In one embodiment, polynucleotides are mixed with cationic lipids that are useful for the introduction of nucleic acid into the cell, including, but not limited to, LIPOFECTIN™ (DOTMA) which consists of a monocationic choline head group that is attached to diacylglycerol (see generally, U.S. Pat. No. 5,208,036 to Epstein et al.); TRANSFECTAM™ (DOGS) a synthetic cationic lipid with lipospermine head groups (Promega, Madison, Wis.); DMRIE and DMRIE.HP (Vical, La Jolla, Calif.); DOTAP™ (Boehringer Mannheim (Indianapolis, Ind.), and Lipofectamine (DOSPA) (Life Technology, Inc., Gaithersburg, Md.).

In other embodiments, the therapeutic bio-inks (i.e., polypeptides, polynucleotides, small molecules, drugs, cells, etc.) may be mixed with or encapsulated into microspheres or nanospheres that promote penetration into mammalian tissues and uptake by mammalian cells. In various embodiments, the microspheres or nanospheres may optionally have other molecules bound to them. These modifications may, for example, impart the microspheres or nanospheres with the ability to target and bind specific tissues or cells, allow them be retained at the administration site, protect incorporated bioactive agents, exhibit antithrombogenic effects, prevent aggregation, and/or alter the release properties of the microspheres. Production of such surface-modified microspheres are discussed in Levy et al., PCT Application No. WO 96/20698, the disclosure of which is hereby incorporated by reference. In exemplary embodiments, it may be desirable to incorporate receptor-specific molecules into or onto the microspheres to mediate receptor-specific particle uptake, including, for example, antibodies such as IgM, IgG, IgA, IgD, and the like, or any portions or subsets thereof, cell factors, cell surface receptors, MHC or HLA markers, viral envelope proteins, peptides or small organic ligands, derivatives thereof, and the like.

In other embodiments, the therapeutic bio-inks (i.e., polypeptides, polynucleotides, small molecules, drugs, cells, etc.) may be mixed or complexed with particulates that promote delivery to, or uptake by mammalian cells, provide osteoconductive properties, influence mass transport, etc. In certain embodiments, suitable particulates include bioceramics such as hydroxyapatite (“HA”) or other calcium containing compounds such as mono-, di-, octa-, alpha-tri-, beta-tri-, or tetra-calcium phosphate, fluoroapatite, calcium sulfate, calcium fluoride and mixtures thereof; bioactive glass comprising metal oxides such as calcium oxide, silicon dioxide, sodium oxide, phosphorus pentoxide, and mixtures thereof; and the like. In an exemplary embodiment, hydroxyapatite is used as the bioceramic material because it provides osteoinductive and/or osteoconductive properties. It is preferable that the particle size of the particulates be about 0.1 nm to about 100 nm, more preferably about 2 nm to about 50 nm.

In various embodiments, the therapeutic bio-inks may be formulated so as to provide controlled release over time, for example, days, weeks, months or years. This may be accomplished by co-deposition with one or more biodegradable structural bio-inks and/or one or more functional bio-inks such that the therapeutic bio-ink is released over time as the biomimetic scaffold is degraded or eroded. In an exemplary embodiment, degradation of the scaffold is modulated by a functional bio-ink that decreases (e.g., via a peptide, protein, or chemical protease, such as, for example, aprotinin) or increases (e.g., a protease) the rate of degradation and/or erosion of the scaffold. Alternatively, the therapeutic bio-inks may comprise a microsphere composition which is attached to or incorporated within the biomimetic scaffold. In this embodiment, the biomimetic scaffold need not degrade in order to produce a time released effect of the therapeutic bio-ink. Release properties can also be determined by the size and physical characteristics of the microspheres.

In other embodiments, the therapeutic bio-inks are incorporated into the biomimetic scaffold or covalently attached to the scaffold during the co-depositing process.

In still other embodiments, the therapeutic bio-inks may be cells. In various embodiments, cells may be sprayed, directly deposited individually, as a population aliquot, or pre-bound, with or without in vitro expansion, to various cell carriers or micro structures. Exemplary cell types include, for example, cells derived from a variety of tissues such as lung, liver, kidney, thymus, thyroid, heart, brain, pancreas (including acinar and islet cells), mesenchymal cells (including bone, cartilage, ligament, tendon, etc.), especially smooth or skeletal muscle cells, myocytes (muscle stem cells), fibroblasts, chondrocytes, adipocytes, fibromyoblasts, and ectodermal cells, including ductile and skin cells, hepatocytes, Islet cells, cells present in the intestine, and other parenchymal cells, osteoblasts and other cells forming bone or cartilage. In some cases it may also be desirable to include nerve cells. Cells can be normal or genetically engineered to provide additional or normal function. Methods for genetically engineering cells with retroviral vectors, polyethylene glycol, or other methods known to those skilled in the art can be used.

Cells are preferably autologous cells, obtained by biopsy and expanded in culture, although cells from close relatives or other donors of the same species may be used with appropriate immunosuppression. Immunologically inert cells, such as embryonic or fetal cells, stem cells, and cells genetically engineered to avoid the need for immunosuppression can also be used. Methods and drugs for immunosuppression are known to those skilled in the art of transplantation. A preferred compound is cyclosporin using the recommended dosages.

In the preferred embodiment, cells to be used as a therapeutic bio-ink are obtained by biopsy and expanded in culture for subsequent implantation. Cells can be easily obtained through a biopsy anywhere in the body, for example, skeletal muscle biopsies can be obtained easily from the arm, forearm, or lower extremities, and smooth muscle can be obtained from the area adjacent to the subcutaneous tissue throughout the body. To obtain either type of muscle, the area to be biopsied can be locally anesthetized with a small amount of lidocaine injected subcutaneously. Alternatively, a small patch of lidocaine jelly can be applied over the area to be biopsied and left in place for a period of 5 to 20 minutes, prior to obtaining biopsy specimen. The biopsy can be effortlessly obtained with the use of a biopsy needle, a rapid action needle which makes the procedure extremely simple and almost painless. With the addition of the anesthetic agent, the procedure would be entirely painless. This small biopsy core of either skeletal or smooth muscle can then be transferred to media consisting of phosphate buffered saline. The biopsy specimen is then transferred to the lab where the muscle can be grown utilizing the explant technique, wherein the muscle is divided into very pieces which are adhered to culture plate, and serum containing media is added. Alternatively, the muscle biopsy can be enzymatically digested with agents such as trypsin and the cells dispersed in a culture plate with any of the routinely used medias. After cell expansion within the culture plate, the cells can be easily passaged utilizing the usual technique until an adequate number of cells is achieved.

In still other embodiments, the therapeutic bio-inks are cells which naturally produce, or have been engineered to produce, a gene product of interest. Such gene products may be used to regulate the growth and/or activity of naturally occurring cells of the host into which the biomimetic scaffold has been implanted. For example, tumor suppressor gene products may be used to regulate proliferation of the host cells. Regulated expression of tumor suppressor gene products are particularly useful for a variety of applications. For example, one may want the host cells to undergo a rapid proliferation phase followed by a production phase where cellular energies are devoted to protein production, or a rapid proliferation phase in vitro followed by regulated growth in vivo (see, for example, U.S. application Ser. No. 08/948,381, filed Oct. 9, 1997, the disclosure of which is incorporated by reference). Tumor suppressor gene products, as used herein, may be intracellular proteins that block the cell cycle at a cell cycle checkpoint by interaction with cyclins, Cdks or cyclin-Cdk complexes, or by induction of proteins that do so. Thus, these tumor suppressor gene products inhibit the cyclin-dependent progression of the cell cycle. Particularly preferred tumor suppressor gene products act on the G1-S transition of the cell cycle. Any tumor suppressor gene product which performs this function, whether known or yet to be discovered, may be utilized. Examples of tumor suppressor genes include p21, p27, p53 (and particularly, the p53175P mutant allele), p57, p15, p16, p18, p19, p73, GADD45 and APC1

In other embodiments, the therapeutic bio-inks may be cells that express survival factors. Survival factors are intracellular proteins that prevent apoptosis such as bcl-2, bcl-x_{L}, E1B-19K, mc1-1, cimA, ab1, p35, bag-1, A20, LMP-1, Tax, Ras, Rel and NF-κB-like factors. Additionally, all known survival factors, as well as survival factors yet to be discovered, are useful in the methods and compositions disclosed herein. In yet another embodiment, the tumor suppressor gene(s) is expressed concomitantly with a factor that stabilizes the tumor suppressor gene product in the cell. Examples of stabilizing factors are members of the CAAT enhancer binding protein family. For example, p21 protein activity is stabilized when coexpressed with C/EBP-alpha. Additionally, C/EBP-alpha specifically induces transcription of the endogenous p21 gene. Thus, C/EBP-alpha functions as both a stabilizing factor and as a specific inducer of p21.

In still other embodiments, the therapeutic bio-inks may be cells that express a gene product that activates cell proliferation. For example, a protein that activates cell proliferation is Mekl, a central protein kinase in the conserved mammalian Ras-MAP signal transduction pathway responding to growth-promoting signals such as cytokines. A particularly preferred version of Mekl is the Mekl DD mutant (Gruelich and Erikson, 1998, J. Biol. Chem. 273: 13280-13288) described more fully below. Other genetic determinants exerting positive control of mammalian cell cycle that can be used as a protein that activates cell proliferation are cyclins (e.g., cyclin E), Ras, Raf, the MAP kinase family (e.g., MAP, Erk, Sap) E2F, Src, Jak, Jun, Fos, pRB, Mek2, EGF, TGF, PDGF, and a polynucleotide that is antisense to a tumor suppressor gene (e.g., p27 antisense expression has been shown to stimulate proliferation of quiescent fibroblasts and enable growth in serum-free medium (Rivard et al., 1996, J. Biol. Chem. 271: 18337-18341) and nedd5 which is known as positive growth controlling gene (Kinoshita et al, 1997, Genes Dev. 11: 1535-1547).

In certain embodiments, the therapeutic bio-inks may be cells that express a transcription factor, such as, for example, RUNX and/or osteogenics. In various embodiments, the cells may either naturally express a transcription factor of interest or may be recombinantly engineered to express a transcription factor of interest.

In yet other embodiments, the therapeutic bio-inks may be cells that contain genes whose expression can be regulated by external factors. For example, an antibiotic-regulated gene expression in eukaryotic cells based on the repressor of a streptogramin resistance operon of S. coelicolor (a Pip) has been described in U.S. Pat. No. 6,287,813. Briefly, a Pip protein (PIT4), or chimeric Pip proteins (PIT and PIT2) fused to a eukaryotic transactivator can be used to control expression of a synthetic eukaryotic promoter (P_{PIR}) containing the Pₚₜᵣ-binding site (in other words, a P_{abr}-linked gene). Genes placed under the control of this PIT/P_{PIR} system are responsive to clinically approved therapeutic compounds belonging to the streptogramin group (pristinamycin, virginiamycin and Synercid) in a variety of mammalian cell lines (CHO-K1, BHK-21 and HeLa). The well-established tetracycline-based system used in conjunction with CHO cells engineered to provide both streptogramin and tetracycline regulation may also be used.

In certain embodiments, therapeutic bio-inks may also include adjuvants and additives, such as stabilizers, fillers, antioxidants, catalysts, plasticizers, pigments, and lubricants, to the extent such ingredients do not diminish the utility of the bio-ink for its intended purpose.

Apparatus

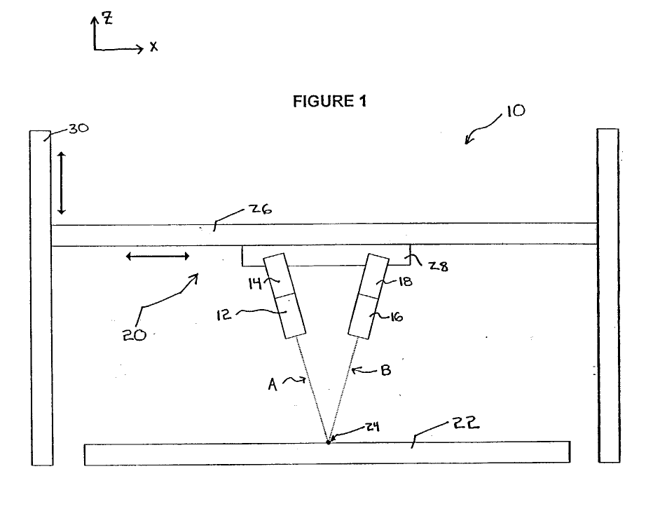

FIG. 1 illustrates an exemplary embodiment of an apparatus for dispensing bio-inks onto a surface. The exemplary apparatus 10 includes a first micro-dispensing device 12 fluidly connected to a source 14 of a first bio-ink and configured to dispense a volume of the first bio-ink and a second micro-dispensing device 16 fluidly connected to a source 18 of a second bio-ink and configured to dispense a volume of the second bio-ink. A movable stage 20 supports the first micro-dispensing device 12 and the second micro-dispensing device 16. In the exemplary embodiment, the movable stage 20 is configured to move the first micro-dispensing device 12 and the second dispensing device 16 relative to a surface 22. During operation, the first micro-dispensing device 12 and the second micro-dispensing device 16 may be displaced by the stage 20 relative to the surface 22 and may selectively dispense a volume of the first bio-ink and a volume of the second bio-ink at a plurality of dispensing locations on the surface 22. The exemplary apparatus 10 is particularly suited for in vitro fabrication of biomimetic structures, in which case the surface may be a slide or other structure suitable for in vitro fabrication of biomimetic structures. The exemplary apparatus 10 is also particularly suited for in situ and (in vivo) fabrication of biomimetic structures, in which case the surface may be a portion of a subject. In one exemplary application, selected bio-inks may be incrementally deposited on the surface in successive layers to fabricate a 3-D scaffold of a biomimetic structure.

In other exemplary applications, the apparatus 10 may be used to dispense bio-inks in vivo to treat a subject. For example, the apparatus 10 may employed to dispense bio-inks onto a surgical site during minimally invasive surgery or other surgical procedures.

The first micro-dispensing device 12 and the second micro-dispensing device 16 may be any device suitable for dispensing small volumes of fluids. Exemplary micro-dispensing devices may include micro-dispensing solenoid valves, ink jet print heads, such as, for example, drop-on-demand piezoelectric ink-jet print heads, and precision syringe pumps. A suitable micro-dispensing valve is available from the Lee Company of Westbrook, Connecticut and a suitable piezoelectric head is available Microfab, Inc. of Plano, Tex. Alternatively, an array of ink-jet print heads, such as an array of jets in banks of 64, such as Model LT-8110 ink jet print heads available from Ink Jet Technology, Inc. of San Jose, Calif., may be employed. Suitable precision syringe pumps are described in detail in U.S. Pat. No. 5,916,524 to Tisone, incorporated herein by reference. The particular micro-dispensing device used in the exemplary apparatus 10 may depend on a number of factors, including the volume of fluid to be dispensed, the desired velocity of the fluid through the micro-dispensing device, and the fluid, e.g., the bio-ink, being dispensed. In certain exemplary embodiments, a suitable micro-dispensing device may dispense fluids in volumes of less than 100 nanoliters. In other exemplary embodiments, a suitable micro-dispensing device may dispense fluids in volumes of less than 100 picoliters. One skilled in the art will appreciate that the first micro-dispensing device 12 and the second micro-dispensing device 16 need not be the same type of micro-dispensing device.

In the exemplary embodiment illustrated in FIG. 1, the first micro-dispensing device 12 and the second micro-dispensing device 16 are each fluidly coupled to a respective source 14, 18 of bio-ink positioned proximate the micro-dispensing device. Each bio-ink source 14, 18, in the exemplary embodiment, may be a reservoir or other container suitable for holding a fluid. Each source 14, 18 may be fluidly coupled by piping or other fluid conduits to provide the bio-ink to a respective micro-dispensing device. The bio-ink may be gravity fed from a source to a micro-dispenser or, alternatively, the bio-ink may be displaced by other mechanisms know in the art for moving fluids, including by compressed gas or by a pumping device, such as a syringe. The bio-ink sources 14, 18 may also be located remotely from the micro-dispensing devices and may be piped or other wise transported to a respective micro-dispensing device. A temperature controlled heat source may be provided with one or both bio-ink sources 14, 18 or with one or both micro-dispensing devices 12, 16 to maintain the bio-ink at a desired temperature. Also, a temperature controlled cooling unit may be provided with one or both bio-ink sources 14, 18 or with one or both micro-dispensing devices 12, 16 to maintain the bio-ink at a desired temperature.

The first bio-ink and second bio-ink may be any of the bio-ink solution described above. Each bio-ink source 14, 18 may contain the same or a different type of bio-ink solution. In embodiments in which the first bio-ink and the second bio-ink are identical, the micro-dispensing devices 12 and 16 may be fluidly connected to a single common bio-ink source.

Although the exemplary embodiment illustrated in FIG. 1 includes two micro-dispensing devices, any number of micro-dispensing devices may be employed depending on the structure being created or the process being performed. For example, in certain embodiments one micro-dispensing device may be employed to dispense a bio-ink onto a surface. In minimally invasive surgery, for example, an apparatus having one micro-dispensing device may be employed to dispense a functional bio-ink, such as a tissue sealant, at a surgical site. In other exemplary embodiments, an apparatus including one micro-dispensing device may be employed to dispense a single structural bio-ink to create a biomimetic structure. Such structural bio-inks may include bio-inks that solidify without the presence of a second bio-ink, including, for example, thermosensitive hydrogels, pH dependent hydrogels, or light sensitive hydrogels.

In the exemplary embodiment illustrated in FIG. 1, the first micro-dispensing device 12 and the second micro-dispensing device 16 are focused to a focal point 24 such that a dispensed volume of the first bio-ink converges with a dispensed volume of the second bio-ink at the focal point 24. Line A and line B schematically illustrate the path of a volume of the first bio-ink dispensed from the first micro-dispensing device 12 (line A) and the path of a volume of the second bio-ink dispensed from the second micro-dispensing device 16 (line B) converging at the focal point 24. The first micro-dispensing device 12 and the second micro-dispensing device 16 may be focused to the focal point 24 by adjusting the orientation of one or both of the micro-dispensing devices. The focal point 24 may be adjusted relative to the surface 22 by moving the first micro-dispensing device 12 and the second micro-dispensing device 16 orthogonal to the surface 22, i.e., along the Z-axis, with the movable stage 20. By maintaining the focal point 24 proximate to or at the surface 24, the first micro-dispensing device 12 and the second micro-dispensing device 16 can operate to selectively dispense a focused volume of the first bio-ink and second bio-ink at a plurality of dispensing locations on the surface 22. In this manner, the first bio-ink and second bio-ink may converge proximate to or at the substrate surface. Upon convergence, the first bio-ink and the second bio-ink may interact with each other, i.e., mix or diffuse.

The movable stage 20 may comprise a system of electronically and/or manually controllable X-Y-Z stages that permit the first micro-dispensing device 12 and the second micro-dispensing device 16 to be moved along the X-axis, Y-axis, and Z-axis relative to the surface. For example, an X-stage 26 may be operable to displace the micro-dispensing devices along the X-axis, a Y-stage 28 operable to displace the micro-dispensing devices along the Y-axis, and a Z-stage 30 may be operable to displace the micro-dispensing devices along the Z-axis. Suitable electronically controllable X-Y-Z stages are available from Parker Hannifin of Wadsworth, Ohio. One skilled in the art will appreciate that other movable stage devices capable of accurate displacement of small distances may alternatively be employed, including, for example, servomechanisms that permit feedback controlled motion along each axis In an alternative embodiment, a movable stage may be provided to move the surface 22 relative to micro-dispensing devices.

In certain embodiments, the micro-dispensing devices 12 and 16 may be adjustable relative to the movable stage 20. For example, one or both of the micro-dispensing devices may be rotatably adjustable such that the direction of discharge from the mirco-dispensing device may be adjusted. Permitting rotatable adjustment facilitates the selective focusing of the mirco-dispensing devices. In this manner, the apparatus 10 can be operated with the first micro-dispensing device 12 and the second micro-dispensing device 16 focused to a common focal point or, alternatively, with the micro-dispensing devices in an unfocused orientation such that the volume of bio-ink or other solution discharged from each micro-dispensing device does not converge at or proximate the surface 22. Moreover, the micro-dispensing devices 12, 16 may be adjustable in the X-, Y-, and/or Z-axis relative to the movable stage 22.

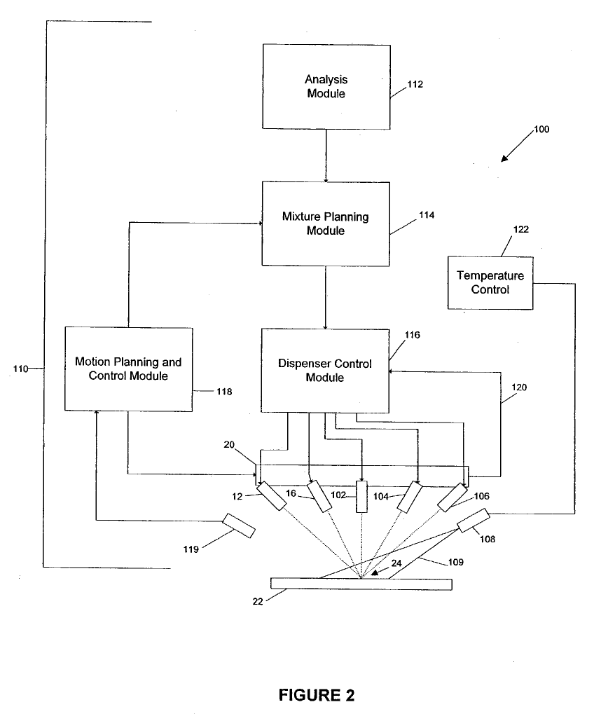

FIG. 2 illustrates another exemplary embodiment of an apparatus for dispensing bio-inks onto a surface. The exemplary apparatus 100 includes a plurality of micro-dispensing devices, including a first micro-dispensing device 12, a second micro-dispensing device 16, a third micro-dispensing device 102, a fourth micro-dispensing device 104, and a fifth micro-dispensing device 106. Any number of micro-dispensing devices may be employed, including one micro-dispensing device. The particular number of micro-dispensing devices provided in the apparatus 100 can be varied depending upon the application. Each of the micro-dispensing devices may be fluidly connected to an independent source of bio-ink or other solution, such as, for example, a buffer solution. Alternatively, one or more of the micro-dispensing devices may be connected to one or more common sources of bio-ink or other fluids.

In the exemplary embodiment illustrated in FIG. 2, each of the micro-dispensing devices may be coupled to the movable stage 20 and may be moved relative to the surface 22. Alternatively, the micro-dispensing devices may be coupled to one or more separate movable stages. As discussed above, a separate movable stage may be provided for the surface 22, as a substitute for movable stage 20 or to complement movable stage 20, in order to move the micro-dispensing devices relative to the surface 20.

One or more of the micro-dispensing devices may be focused to a common focal point such that a volume of bio-ink or other solution dispensed from one of the focused micro-dispensing devices will converge at the focal point with a volume of bio-ink or other solution dispensed from one or more of the other focused micro-dispensing devices. In the exemplary embodiment illustrated in FIG. 2, each of the micro-dispensing devices is focused to a common focal point 24.

The apparatus 100 may also include a heat source 108 for heating at least a portion of the surface 22. In certain embodiments, heating the dispensing locations on the substrate may facilitate the interaction of the deposited bio-inks and/or may inhibit degradation of the dispensed bio-inks. In the exemplary embodiment illustrated in FIG. 2, the heat source 108 may be a light source, such as an infrared light source, that illuminates a portion of the surface 20 with infrared light 109. Alternative heat sources may also be employed, including, for example, one or heating elements attached to or incorporated in a structure supporting the surface.

The exemplary apparatus 100 may also include a control system 110 that is connected to one or more of the plurality of micro-dispensing devices to control the volume of bio-ink or other solution dispensed by the micro-dispensing devices. In the exemplary embodiment, the control system 100 includes multiple modules for effecting control over the solid freeform fabrication of a biomimetic scaffold by controlling the volume of bio-ink dispensed by each micro-dispensing device and the location of the micro-dispensing devices relative to the surface 22. Each of the modules may be instructions for causing a processor to execute the specified features of the module. The instructions and/or modules may be implemented in one or more processors. The processors can be connected over a wireless or wired communication link.

For example, the control system 110 may have an analysis module 112 configured to analyze a 3-D computer generated model of the biomimetic scaffold to determine the composition and/or properties of the scaffold. Properties of the scaffold determined by the analysis module 112 may include the mechanical properties, e.g., the porosity, of scaffold. The volumetric concentration of any therapeutic or functional bio-inks of the scaffold at particular 3-D locations in the scaffold may be determined. The analysis module 112 may be configured to subdivide the computer generated 3-D model into discrete cube units, referred to as voxels. The three-dimension model may then be divided into layers of voxels. The number of layers may be dependent on the resolution of the micro-dispensing devices employed by the apparatus 100. For example, the model may be divided into a greater number of layers as the resolution of the apparatus 100 increases. The analysis module 112 may determine the composition and properties of each of the voxels. For example, the mechanical properties of each voxel and the volume concentration of any therapeutic or functional bio-inks may be determined. The analysis module 112 may utilize any 3-D modeling tool useful in computer added design (CAD). Suitable 3-D modeling tools may include the 3-D ACIS Modeler available from Spatial Corporation of Westminster, Colo.

The control system 110 may include a mixture-planning module 114 configured to determine a volume of bio-ink and/or other solution to be dispensed in each voxel based on the properties and/or composition of the each voxel. In one exemplary embodiment, the total volume of fluid deposited in each voxel is held constant. For example, if the volume concentration of one bio-ink is reduced for a voxel, the volume concentration of another bio-ink or solution may be increased to maintain a constant total volume for the voxel. The volume of each bio-ink and/or other solution to be dispensed for each voxel may be encoded as gray-level values and stored in image buffers provided with the mixture-planning module. In one embodiment, separate image buffers may be provided for each micro-dispensing device.

Continuing to refer to FIG. 2, the exemplary control system 110 may include a dispenser control module 116 that is connected by wireless or wired communication links to one or more of the micro-printing devices. The dispenser control module 116 is configured to provide control signals to one or more of the micro-dispensing devices to control the volume of bio-ink and/or solution dispensed based upon the volumes determined by the mixture-planning module 114. The dispenser control module 116 may comprise one or more processors such as a programmable logic controller (PLCs), for example, a MELSEC-Q series PLC available from Mitsubishi. Alternatively, the dispenser control module 116 may comprise one or more digital signal processors (DSPs), such as, for example, TMS530 series DSPs from Texas Instruments.

The dispenser control module 116 is connected to the mixture-planning module 114 and receives the gray-level values from the image buffers of the mixture-planning module 114 and synthesizes waveforms to drive the micro-dispensing devices. In certain exemplary embodiments, a general-purpose microprocessor or programmable function generator, such as a programmable pulse generator, may be programmed to synthesize waveforms to drive the micro-dispensing devices. In another exemplary embodiment, a separate processor, e.g., a separate programmable logic controller (PLC) or digital signal processor (DSP), is provided for each micro-dispensing device and each processor receives gray-level values from a particular image buffer of the mixture-planning module 114. The waveforms synthesized by the dispenser control module 116 control each micro-dispensing device such that the net volume of fluid dispensed at each dispensing location may be dependent on the droplet volume and the number of droplets dispensed. The droplet volume is dependent on a number of parameters, such as the diameter of the exit nozzle of the micro-dispensing device and the viscosity of the bio-ink or other solution being dispensed. The droplet volume may be adjusted by modulating the waveform of the particular micro-dispensing device. In certain exemplary embodiments, the droplet volume may be frequency controlled by voltage controlled oscillation of the micro-dispensing device. In certain exemplary embodiments, the droplet volume may be controlled by controlling the pressure and on-time of the micro-dispensing device.

The exemplary control system 110 may include a motion planning and control module 118 that is connected by wireless or wired communication link to the movable stage 20. The motion planning and control module 118 is also connected to the mixture-planning module 114. The motion planning and control module 118 controls the motion of the micro-dispensing devices relative to the surface 20. The motion planning and control module 118 may store instructions for one or more deposition strategies. A deposition strategy may specify the sequence in which voxels are deposited and the timing between depositions. For example, one deposition strategy may be to deposit every other voxel in a layer in one pass over the surface 22 and in a second pass over the surface 22, deposit the remaining voxels. Alternatively, a deposition strategy may specify that one or more bio-inks or solutions are deposited for a layer in a first pass and additional bio-inks are deposited in one or more subsequent passes. Another deposition strategy may include dispensing bio-ink in a circumferential pattern. For example, bio-inks may be deposited in a plurality of circular passes, with, for example, each pass creating a layer of bio-ink in a circular pattern. Subsequent circular passes may result in a plurality of concentric circular layers of bio-ink that form one layer of bio-ink. The circular layers may be deposited in sequence, e.g., one circular pass adjacent a previous circular pass. Alternatively, the circular layers may be deposited in radially spaced-apart circular passes to allow bio-ink deposited in one circular pass to set or gel before depositing bio-ink adjacent thereto.

Once a deposition strategy is specified, the strategy is communicated to the mixture-planning module 114 and the stage 20. For example, in the case of a movable stage comprising linear stages for moving the micro-dispensing devices and/or the substrate along the X-, Y-, and Z-axis, the motion planning and control module 118 sets the raster trajectory parameters of each linear stage, including the distance, speed, and line spacing. Encoders or position sensors may provide location feedback along line 120 to the dispenser control module 116 to synchronize the dispensing of bio-inks and/or other solutions with the motion of the micro-dispensing devices relative to the surface 22. One or more depth sensors 119 may be provided to measure the depth of the bio-ink deposited on the surface 22. In applications in which bio-ink is dispensed into a wound or defect, the one or more depth sensors may be used to measure the depth of the wound or defect prior to deposition. Depth measurements may be provided on a layer-by-layer basis, e.g., one or more depth measurements may be taken after the deposition of a layer of bio-ink. Alternatively, depth measurements may be taken continuously and provided to the motion planning control module 118 in a feedback control manner, in which case the micro-dispensing devices may be moved along the Z-axis relative to the stage in response to depth measurements. Suitable depth sensors, include, but are not limited to, optical sensors, acoustic sensors, and touch sensors. In one exemplary embodiment, the depth sensor 119 may be a confocal displacement sensor such as Model LT-8110 available from Keyenece Corp. of America (Beachwood, Ohio).

The exemplary control system 110 may also include a temperature controller 122 connected by wireless or wired communication link to the heat source 108. The temperature controller 122 may be connected to heating and/or cooling units provided to heat or cool bio-ink or other solution within the micro-dispensing devices or the sources of bio-inks. The temperature controller 122 may control the heat source 108 and any heating and/or cooling units to maintain the temperature of the source 120 and the units within a desired range. One or more temperature sensors may be provided to monitor the temperature of the surface 22 and/or the bio-ink or other solution within the micro-dispensing device and/or sources. The temperature sensors can provide feedback to the temperature controller 122 and may facilitate control of the heat source 108 and/or the heating and cooling units.

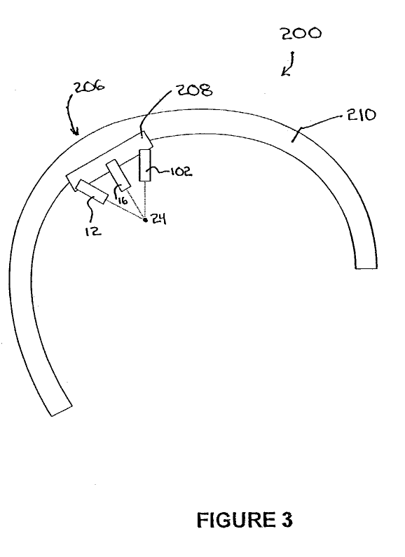

FIG. 3 illustrates an exemplary embodiment of an apparatus for in situ dispensing of a bio-ink or other solution on a subject. The exemplary apparatus 200 may include a first micro-dispensing device 12, a second micro-dispensing device 16, and a third micro-dispensing device 102, although a number of micro-dispensing devices may be employed, including a single micro-dispensing device. Each of the micro-dispensing devices may be fluidly connected to a source of bio-ink or other solution and may dispense a volume of bio-ink as discussed above. The micro-dispensing devices may be connected to a movable stage 208 that may be affixed to a subject. In the exemplary embodiment, the movable stage 208 is coupled to a stereotactic device 206 that is configured for connection to a human head. Other stereotactic devices may be employed, including stereotactic devices for use with other species, including, for example, rat stereotactic devices. The movable stage may be connected to other devices suitable for connecting a medical device or instrument to a subject. The movable stage 208 is movably connected to the frame 210 of the stereotactic device 206 such that movable stage 208 may move relative to the frame of the stereotactic device and, thus, relative to the subject to which the stereotactic device is affixed. The exemplary apparatus 200 may be particularly suited for in situ fabrication of a biomimetic scaffold to, for example, repair a surgical or traumatic wound or defect in the subject's skull.

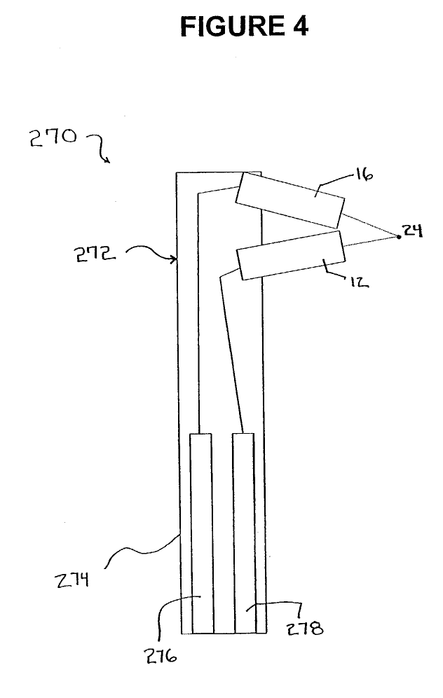

FIG. 4 schematically illustrates an exemplary embodiment of a hand held instrument 270 comprising an instrument frame 272 having a handle sized and shaped to be held by a user and first and second micro-dispensing devices 12 and 16 that are coupled to the frame. In the illustrated embodiment, two focused micro-dispensing devices are illustrated, however, any number of micro-dispensing devices may be employed, including one micro-dispensing device, in a focus or an unfocused relationship. The micro-dispensing devices 12 and 16 may be fluidly connected a first reservoir 276 and a second reservoir 278, respectively. The first reservoir 276 and the second reservoir 278 may contain a source of bio-ink or other solution for a respective mirco-dispensing device. In the exemplary embodiment illustrated in FIG. 4, the first and second reservoirs 276 and 278 are positioned in the instrument frame 272, and in particular, within the handle 274 of the instrument. In alternative embodiments, the micro-dispensing devices may be fluidly connected to remote reservoirs or fluid sources not incorporated in the instrument frame 272. A source of pressurized gas, such as a cartridge of CO2 or other inert gas, may be employed to dispense bio-ink from the reservoirs. The hand-held instrument 270 may be used for in situ and in vivo dispensing of one or more bio-inks. In certain exemplary embodiments, the hand-held sensor may include position, including depth, sensors, temperature sensors, or other sensors for monitoring the dispensing of bio-inks on a surface.

In certain exemplary embodiments, a surgical instrument, such as a minimally invasive surgical instrument, or other medical device may include one or more micro-dispensing devices for dispensing a bio-ink in vivo. For example, minimally invasive surgical instruments for grasping, manipulating, cutting, boring, cauterizing, heating, illuminating, viewing or otherwise treating a subject may include one or micro-dispensing devices for dispensing a bio-ink. In certain exemplary embodiments, robot-assisted surgical devices and systems may include one or more micro-dispensing devices for dispensing a bio-ink in vivo. Certain exemplary robot-assisted surgical devices and systems are described in U.S. Pat. Nos. 5,841,950; 5,855,583; 5,878,913; 6,102,850; 6,233,504; 6,325,808; 6,331,181; and 6,385,509. The afore-mentioned patents are incorporated herein by reference.

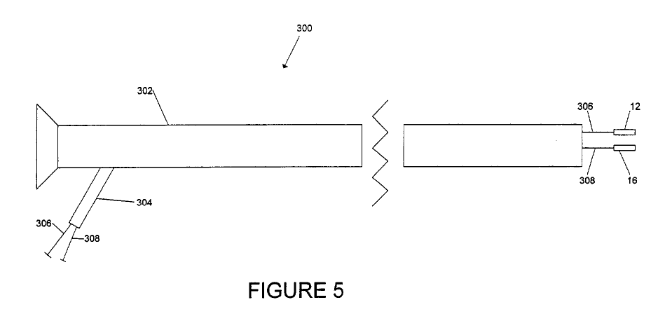

FIG. 5 illustrates an exemplary embodiment of a surgical instrument, a endoscopic apparatus 300, for endoscopic or laparoscopic dispensing of a bio-ink to a subject in vivo. The apparatus 300 comprises an endoscope 302 that is sized and shaped for insertion into a body lumen, organ, or cavity and includes a central instrument lumen 304 through which endoscopic instruments may be delivered. The endoscopic apparatus 300 includes first and a second micro-dispensing devices 12, 16 that may be delivered to the subject through the instrument channel 304 of the endocsope 302. Although two micro-dispensing devices are illustrated, any number of micro-dispensing devices may be employed. The micro-dispensing devices may be coupled to one or more sources of bio-ink or other fluid external to the apparatus 300 by fluid conduits 306 and 308. Each micro-dispensing device 12, 16 may be sized and shaped for insertion through the instrument lumen 304 of the endoscope 302. Other endoscopic instruments, such as a camera or imaging device, may be employed with the endoscopic apparatus 300. The endoscopic apparatus allows the dispensing of bio-inks within a body lumen, organ, or cavity during laparoscopic or endoscopic procedures.

Exemplary Uses

Disclosed herein are systems, compositions and methods useful for making and using biomimetic scaffolds, which may be implanted or created in situ at a desired location. The biomimetic scaffolds disclosed herein may be used to prepare a biomimetic scaffold for any mammal in need thereof. Mammals of interest include humans, dogs, cows, pigs, cats, sheep, horses, and the like, preferably humans.

The methods, compositions, and apparatus disclosed herein may be used to prepare a variety of biomimetic scaffolds that may be utilized as xenografts, allografts, artificial organs, or other cellular transplantation therapeutics. The biomimetic scaffolds may be used to repair and/or replace any damaged tissue associated with a host. The biomimetic scaffolds dislcosed herein may also be suitable for other applications, such as for hormone producing or tissue producing biomimetic implants for deficient individuals who suffer from conditions such as diabetes, thyroid deficiency, growth hormone deficiency, congenital adrenal hyperplasia, Parkinson's disease, and the like. Likewise, apparatus and methods disclosed herein may be useful for creating biomimetic scaffolds suitable for therapeutic applications, including, for example, implantable delivery systems providing biologically active and gene therapy products. For example, the biomimetic scaffolds disclosed herein may be usefully for the treatment of central nervous system, to provide a source of cells secreting insulin for treatment of diabetes, cells secreting human nerve growth factors for preventing the loss of degenerating cholinergic neurons, satellite cells for myocardial regeneration, striatal brain tissue for Huntington's disease, liver cells, bone marrow cells, dopamine-rich brain tissue and cells for Parkinson's disease, cholinergic-rich nervous system for Alzheimer's disease, adrenal chromaffin cells for delivering analgesics to the central nervous system, cultured epithelium for skin grafts, and cells releasing ciliary neurotrophic factor for amyotrophic lateral sclerosis, and the like. In an exemplary embodiment, the biomimetic scaffolds disclosed herein may be used to repair bone injuries and induce healing thereof by inducing vascularization to the site of injury.

In other exemplary embodiments, the methods, compositions and apparatus disclosed herein may be used to create 3-D biomimetic scaffolds capable of providing a spatial and/or temporally organized therapeutic to a host at a desired location. In such embodiments, the scaffolds contain 3-D patterns of therapeutic bio-inks that provide a therapeutic to a host in a predictable and organized manner. For example, a biomimetic scaffold may have gradients of one or more growth factors which vary throughout the structure, such as a concentration gradient that diminishes from the center of the structure to the periphery, a gradient from one side of the structure to the other, etc., in an infinite variety of possible configurations. In addition to spatial gradients, temporal gradients may also be engineered using the time release mechanisms described herein. Using such spatial and/or temporal gradients, organized doses of one or more therapeutic factors can be provided to an organism in need thereof. For example, such spatial and temporal therapeutics may be used to induce organized neovascularization in a host at a desired location. During wound healing, angiogenic factors are produced at the site of injury producing a concentration gradient which decreases away from the site of injury. However, traditional approaches to inducing angiogenesis involve uniform application of angiogenic factors which typically lead to unorganized vessel formations or angiomas. The biomimetic scaffolds disclosed herein may be engineered so as to provide a concentration gradient of angiogenic factors in a 3-D spatial and/or temporal configuration that mimics the naturally occurring wound healing response signals resulting in formation of organized and directed neovascularization at a desired location in a host.

In another embodiment, biomimetic scaffolds may contain a 3-D pattern of adhesion molecules specific for one or more cell types. For example, a 3-D pattern of adhesion molecules may be configured so as to attract and adhere particular cell types to the scaffold in a desired 3-D architecture. These scaffolds can be used to induce a desired configuration of cell attachment/tissue formation at a specified location. The biomimetic scaffold may be a permanent or long-term implant or may degrade over time as the host's natural cells replace the scaffold. In an exemplary embodiment, two or more adhesion molecules with different cell binding specificities are patterned on the biomimetic scaffold so as to immobilize two or more desired cell types into a specific 3-D pattern. In practicing this exemplary embodiment, a variety of techniques can be used to foster selective cell adhesion of two or more cell types to the scaffold. For example, adhesion proteins such as collagen, fibronectin, gelatin, collagen type IV, laminin, entactin, and other basement proteins, including glycosaminoglycans such as heparan sulfate, RGD peptides, ICAMs, E-cadherins, and antibodies that specifically bind a cell surface protein (for example, an integrin, ICAM, selectin, or E-cadherin). Also envisioned are methods such as localized protein adsorption, organosilane surface modification, alkane thiol self-assembled monolayer surface modification, wet and dry etching techniques for creating 3-D substrates, radiofrequency modification, and ion-implantation (Lom et al., 1993, J. Neurosci. Methods 50:385-397; Brittland et al., 1992, Biotechnology Progress 8:155-160; Singhvi et al., 1994, Science 264:696-698; Singhvi et al., 1994, Biotechnology and Bioengineering 43:764-771; Ranieri et al., 1994, Intl. J. Devel. Neurosci. 12(8):725-735; Bellamkonda et al., 1994, Biotechnology and Bioengineering 43:543-554; and Valentini et al., 1993, J. Biomaterials Science Polymer Edition 5(1/2): 13-36).

In still other embodiments, the therapeutic bio-inks disclosed herein may be cells which may be used to directly create a 3-D cellular architecture of one or more cell types. Combinations of these approaches are also envisioned, e.g., 3-D patterns of cells and growth factors. In other embodiments, cells may be used to coat small or large surface areas of devices, wound dressings or areas of the body. Such coatings may be applied directly to the device, wound or region of the body or may be pre-fabricated and applied to a desired location. In various embodiments, cells may be applied individually or as a population aliquot.

In certain embodiments, the apparatus, methods, and compositions described herein may be used to create interpenetrating polymer networks (IPNs). IPNs are blends or alloys of two or more polymer components, each of which is a crosslinked 3-D network. The individual polymer component networks are more or less physically entangled with, but not covalently bonded to the other polymer network(s) in the IPN. A feature of IPNs is that they permit combining advantageous properties from each of two polymers which are normally incompatible. For example, in a hydrophobic-hydrophilic system, flexibility and structural integrity might be imparted by the hydrophobic polymer and lubriciousness might be imparted by the hydrophilic polymer. An IPN may be a bicontinuous system in which each of the polymers forms a continuous matrix throughout the network.

In another embodiment, the apparatus, methods, compositions and products disclosed herein may be used in association with minimally invasive surgery techniques. For example, a biomimetic scaffold may be created in situ, or may be pre-fabricated and implanted into a patient, at a desired location using minimally invasive techniques. In certain embodiments, minimally invasive surgical techniques may be used to provide tissue sealants at focused areas and/or to provide short term and/or long term administration of a therapeutic agents, including for example, therapeutic bio-inks such as cells, polypeptides, polynucleotides, growth factors, drugs, etc. In one exemplary embodiment, minimally invasive techniques may be used to provide biomimetic scaffolds for repairing hyaline cartilage and/or fibrocartilage in diarthroidal and amphiarthroidal joints. In another exemplary embodiment, a resorbable vascular wound dressing may be delivered in association with angioplasty procedures to deliver or fabricate a biomimetic scaffold to selected sites inside or outside a blood vessel. Vascular wound dressings may be tubular, compliant, self-expandable, low profile, biocompatible, hemocompatible and/or bioresorbable. In certain embodiments, such wound dressings may prevent or substantially reduce the risk of post-angioplasty vessel reclosure. In other embodiments, vascular would dressings may be fabricated with a therapeutic bio-ink suitable for treatment of vessel wounds, including, for example, anti-platelet agents such as aspirin and the like, anti-coagulant agents such as coumadin and the like, antibiotics, anti-thrombus deposition agents such as polyanionic polysaccharides including heparin, chondroitin sulfates, hyaluronic acid and the like, urokinase, streptokinase, plasminogin activators and the like, wound healing agents such as transforming growth factor beta (TGF beta) and the like, glycoproteins such as laminin, fibronectin and the like, various types of collagens.

In another embodiment, the apparatus, methods, compositions and products disclosed herein may be used to create bioresorbable wound dressings or band-aids. Wound dressings may be used as a wound-healing dressing, a tissue sealant (i.e., sealing a tissue or organ to prevent exposure to a fluid or gas, such as blood, urine, air, etc., from or into a tissue or organ), and/or a cell-growth scaffold. In various embodiments, the wound dressing may protect the injured tissue, maintain a moist environment, be water permeable, be easy to apply, not require frequent changes, be non-toxic, be non-antigenic, maintain microbial control, and/or deliver effective healing agents to the wound site.

Examples of bioresorbable sealants and adhesives that may be used in accordance with the apparatus, methods, compositions described herein include, for example, FOCALSEAL produced by Focal; BERIPLAST produced by Adventis-Bering; VIVOSTAT produced by ConvaTec (Bristol-Meyers-Squibb); SEALAGEN produced by Baxter; FIBRX produced by CyoLife; TISSEEL AND TISSUCOL produced by Baxter; QUIXIL produced by Omrix Biopharm; a PEG-collagen conjugate produced by Cohesion (Collagen); HYSTOACRYL BLUE produced by Davis & Geck; NEXACRYL, NEXABOND, NEXABOND S/C, and TRAUMASEAL produced by Closure Medical (TriPoint Medical); OCTYL CNA produced by Dermabond (Ethicon); TISSUEGLU produced by Medi-West Pharma; and VETBOND produced by 3M.

Wound dressings may be used in conjunction with orthopedic applications such as bone filling/fusion for osteoporosis and other bone diseases, cartilage repair for arthritis and other joint diseases, and tendon repair and for soft tissue repair, including nerve repair, organ repair, skin repair, vascular repair, muscle repair, and ophthalmic applications. In exemplary embodiments, wound dressings may be used to treat a surface such as, for example, a surface of the respiratory tract, the meninges, the synovial spaces of the body, the peritoneum, the pericardium, the synovia of the tendons and joints, the renal capsule and other serosae, the dermis and epidermis, the site of an anastomosis, a suture, a staple, a puncture, an incision, a laceration, or an apposition of tissue, a ureter or urethra, a bowel, the esophagus, the patella, a tendon or ligament, bone or cartilage, the stomach, the bile duct, the bladder, arteries and veins.

In exemplary embodiments, wound dressings may be used in association with any medical condition that requires coating or sealing of a tissue. For example, lung tissue may be sealed against air leakage after surgery; leakage of blood, serum, urine, cerebrospinal fluid, air, mucus, tears, bowel contents or other bodily fluids may be stopped or minimized; barriers may be applied to prevent post-surgical adhesions, including those of the pelvis and abdomen, pericardium, spinal cord and dura, tendon and tendon sheath. Wound dressings may also be useful for treating exposed skin, in the repair or healing of incisions, abrasions, burns, inflammation, and other conditions requiring application of a coating to the outer surfaces of the body. Wound dressings may also be useful for applying coatings to other body surfaces, such as the interior or exterior of hollow organs, including blood vessels. Restenosis of blood vessels or other passages may also be treated.

The range of uses for wound dressings also include cardiovascular surgery applications, prevention of bleeding from a vascular suture line; support of vascular graft attachment; enhancing preclotting of porous vascular grafts; stanching of diffuse nonspecific bleeding; anastomoses of cardiac arteries, especially in bypass surgery; support of heart valve replacement; sealing of patches to correct septal defects; bleeding after sternotomy; and arterial plugging; thoracic surgery applications, including sealing of bronchopleural fistulas, reduction of mediastinal bleeding, sealing of esophageal anastomoses, and sealing of pulmonary staple or suture lines; neurosurgery applications, including dural repairs, microvascular surgery, and peripheral nerve repair; general surgery applications, including bowel anastomoses, liver resection, biliary duct repair, pancreatic surgery, lymph node resection, reduction of seroma and hematoma formation, endoscopy-induced bleeding, plugging or sealing of trocar incisions, and repair in general trauma, especially in emergency procedures; plastic surgery applications, including skin grafts, burns, debridement of eschars, and blepharoplasties (eyelid repair); otorhinolaryngology (ENT) applications, including nasal packing, ossicular chain reconstruction, vocal cord reconstruction and nasal repair; opthalmology applications, including corneal laceration or ulceration, and retinal detachment; orthopedic surgery applications, including tendon repair, bone repair, including filling of defects, and meniscus repairs; gynecology/obstetrics applications, including treatment of myotomies, repair following adhesiolysis, and prevention of adhesions; urology applications, including sealing and repair of damaged ducts, and treatment after partial nephrectomy are potential uses; dental surgery applications, including treatment of periodontal disease and repair after tooth extraction; repair of incisions made for laparoscopy or other endoscopic procedures, and of other openings made for surgical purposes, are other uses; treatment of disease conditions such as stopping diffuse bleeding in hemophiliacs; and separation of tissues to prevent damage by rubbing during healing. In each case, appropriate therapeutic agents may be included in the wound dressing.

In certain embodiments, wound dressings may be fabricated with therapeutic bio-inks to provide delivery of a therapeutic agent at a site of injury, including, for example, anti-infectives such as antibiotic, anti-fungal or anti-viral agents, anti-inflammatory agents, mitogens to stimulate cell growth and/or differentiation, agents to stimulate cell migration to the site of injury, growth factors, cells such as osteoblasts, chondrocytes, keratinocytes, and hepatocytes, to restore or replace bone, cartilage, skin, and liver tissue respectively, etc. Alternatively, therapeutic agents may be added to the wound dressing after fabrication, e.g., by soaking, spraying, painting, or otherwise applying the therapeutic agent to the dressing.

In various embodiments, wound dressings may be fabricated directly at a desired location or may be pre-fabricated and applied to the wound. Wound dressings may be in the form of flat films that may be adhered to tissue to cover the site of an injury or may be in the form of 3-D structures such as plugs or wedges. Pre-fabricated wound dressings may be supplied in standard configurations suitable for application to a variety of wounds and may be applied as is or may be cut, molded or otherwise shaped prior to application to a particular wound. Alternatively, pre-fabricated wound dressings may be produced in a configuration tailored to a specific wound or wound type. In one embodiment, the wound dressing is supplied as a moist material that is ready for application to a wound. In another embodiment, the wound dressing is supplied as a dried material which may be rehydrated upon or prior to application to a wound.

In another embodiment, the apparatus, methods, compositions and products disclosed herein may be used to fabricate coatings for devices to be used in the body or in contact with bodily fluids, such as medical devices, surgical instruments, diagnostic instruments, drug delivery devices, and prosthetic implants. Coatings may be fabricated directly on such objects or may be pre-fabricated in sheets, films, blocks, plugs, or other structures and applied/adhered to the device. Such coating may be useful as a tissue-engineering scaffold, as a diffusion membrane, as a method to adhere the implant to a tissue, as a delivery method for a therapeutic agent, and/or as a method to prolong implant stability, e.g., by preventing or suppressing an immune response to the implant from the host. In various embodiment, coatings may be applied to implantable devices, such as pacemakers, defibrillators, stents, orthopedic implants, urological implants, dental implants, breast implants, tissue augmentations, heart valves, artificial corneas, bone reinforcements, and implants for maxillofacial reconstruction; devices such as percutaneous catheters (e.g. central venous catheters), percutaneous cannulae (e.g. for ventricular assist devices), catheters, urinary catheters, percutaneous electrical wires, ostomy appliances, electrodes (surface and implanted), and supporting materials, such as meshes used to seal or reconstruct openings; and other tissue-non-tissue interfaces.

In an exemplary embodiment, a bio-ink may be printed directly into a seeping wound to seal off the blood flow and provide a clear printing area. Such wound plug or blood clotting applications may be particularly useful, for example, in battlefield applications.

In certain embodiments, wound dressings may be fabricated with therapeutic bio-inks to provide delivery of a therapeutic agent at a desired location. Therapeutic agents may be included in a coating as an ancillary to a medical treatment (for example, antibiotics) or as the primary objective of a treatment (for example, a gene to be locally delivered). A variety of therapeutic agents may be used, including passively functioning materials such as hyaluronic acid, as well as active agents such as growth hormones. A wide variety of therapeutic agents may be used, including, for example, cells, proteins (including enzymes, growth factors, hormones and antibodies), peptides, organic synthetic molecules, inorganic compounds, natural extracts, nucleic acids (including genes, antisense nucleotides, ribozymes and triplex forming agents), lipids and steroids, carbohydrates (including heparin), glycoproteins, and combinations thereof. The agents to be incorporated can have a variety of biological activities, such as vasoactive agents, neuroactive agents, hormones, anticoagulants, immunomodulating agents, cytotoxic agents, antibiotics, antivirals, or may have specific binding properties such as antisense nucleic acids, antigens, antibodies, antibody fragments or a receptor.

In exemplary embodiments, therapeutic agents which may be used in conjunction with a coating include antibiotics, antivirals, anti-inflammatories, both steroidal and non-steroidal, anti-neoplastics, anti-spasmodics including channel blockers, modulators of cell-extracellular matrix interactions including cell growth inhibitors and anti-adhesion molecules, enzymes and enzyme inhibitors, anticoagulants and/or antithrombotic agents, growth factors, DNA, RNA, inhibitors of DNA, RNA or protein synthesis, compounds modulating cell migration, proliferation and/or growth, vasodilating agents, and other drugs commonly used for the treatment of injury to tissue. Specific examples of these compounds include angiotensin converting enzyme inhibitors, prostacyclin, heparin, salicylates, nitrates, calcium channel blocking drugs, streptokinase, urokinase, tissue plasminogen activator (TPA) and anisoylated plasminogen activator (TPA) and anisoylated plasminogen-streptokinase activator complex (APSAC), colchicine and alkylating agents, and aptamers. Specific examples of modulators of cell interactions include interleukins, platelet derived growth factor, acidic and basic fibroblast growth factor (FGF), transformation growth factor .beta. (TGF -beta), epidermal growth factor (EGF), insulin-like growth factor, and antibodies thereto. Specific examples of nucleic acids include genes and cDNAs encoding proteins, expression vectors, antisense and other oligonucleotides such as ribozymes which can be used to regulate or prevent gene expression. Specific examples of other bioactive agents include modified extracellular matrix components or their receptors, and lipid and cholesterol sequestrants.

In further embodiments, therapeutic agents which may be used in conjunction with a coating include proteins, such as cytokines, interferons and interleukins, poetins, and colony-stimulating factors. Carbohydrates including Sialyl Lewis which has been shown to bind to receptors for selectins to inhibit inflammation. A ‘Deliverable growth factor equivalent’ (abbreviated DGFE), a growth factor for a cell or tissue, may be used, which is broadly construed as including growth factors, cytokines, interferons, interleukins, proteins, colony-stimulating factors, gibberellins, auxins, and vitamins; further including peptide fragments or other active fragments of the above; and further including vectors, i.e., nucleic acid constructs capable of synthesizing such factors in the target cells, whether by transformation or transient expression; and further including effectors which stimulate or depress the synthesis of such factors in the tissue, including natural signal molecules, antisense and triplex nucleic acids, and the like. Exemplary DGFE's are VEGF, ECGF, bFGF, BMP, and PDGF, and DNA's encoding for them. Exemplary clot dissolving agents are tissue plasminogen activator, streptokinase, urokinase and heparin.

In other embodiments, drugs having antioxidant activity (i.e., destroying or preventing formation of active oxygen) may be used, which are useful, for example, in the prevention of adhesions. Examples include superoxide dismutase, or other protein drugs include catalases, peroxidases and general oxidases or oxidative enzymes such as cytochrome P450, glutathione peroxidase, and other native or denatured hemoproteins.

In still other embodiments, mammalian stress response proteins or heat shock proteins, such as heat shock protein 70 (hsp 70) and hsp 90, or those stimuli which act to inhibit or reduce stress response proteins or heat shock protein expression, for example, flavonoids, also may be used.

Characterization of the Biomimetic Structure

The biomimetic structures disclosed herein may be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

The microstructure (porosity, fibril diameter) of biomimetic structures may be characterized using scanning electron microscopy (SEM) and fluorescence confocal microscopy. Patterns and concentrations of therapeutic factors may be determined by fluorescence microscopy using direct fluorescent labeling and immunofluorescence.

### EXAMPLES

The apparatus, methods and compositions disclosed herein now being generally described, it will be more readily understood by reference to the following examples which are included merely for purposes of illustration of certain aspects and embodiments of the present disclosure, and are not intended to limit the present disclosure in any way.

In an exemplary embodiment, an innovative scaffold fabrication process may be used in situ or ex vivo to manufacture a tissue engineered therapy to control angiogenesis. The process may be used to fabricate a biomimetic fibrin extracellular matrix (bECM) incorporating a patterned 3-D solid-phase (i.e., cross-linked to the matrix) concentration gradient of recombinant human fibroblast growth factor-2 (FGF-2).

In general, bECM with a patterned spatial distribution of FGF-2 may be used to induce controlled angiogenesis. In particular, a fibrin-based bECM design with gradients of FGF-2 targeted for angiogenesis is used in bone tissue engineering. Angiogenesis is a requisite for osteogenesis and successful incorporation of tissue engineered bone grafts. A broad range of native matrix materials and components targeted for different tissues may be applicable.

There are a several strategies to address angiogenesis in engineered tissue constructs. Most often, a bECM delivery of growth factors (GFs), cells or both, provide structural support, cues, and surfaces for cell attachment. Examples include seeding and culturing bECMs with ECs and other cells in vitro seeding and culturing structured bECMs, which have intrinsic networks of channels, with hepatocytes and other cell types in vitro seeding ECs and other cells into micromachined branched channels, cultured in vitro, and the resulting layers are folded into 3-D structures; seeding bECMs with cells transfected with a recombinant retrovirus encoding VEGF; and incorporation of VEGF-A165 or FGF-2 in bECMs by entrapment, adsorption, microcarriers or immobilized to matrices by covalent bonding. In an exemplary embodiment, a process of forming a biomimetic scaffold includes printing fibrin bECMs in situ with defined solid-phase 3-D patterns of FGF-2. This process may provide a controlled and predictable angiogenic response.

Wound Healing Biology and Angiogenesis. Cells, GFs, and an ECM are fundamental tissue building blocks. Functional roles for each of these building blocks in homeostasis and wound repair guide the tissue engineering designs. Angiogenesis is a reoccurring theme in homeostasis and wound repair. As a consequence of the powerful role angiogenesis has on wound repair, the apparatus, compositions, and methods disclosed herein provide for tissue-engineered therapies. Without angiogenesis, tissues with a volume exceeding a few cubic millimeters cannot survive by diffusion of nutrients and oxygen.

Angiogenesis occurs under specific spatial and temporal control. It has been suggested that temporal release of VEGF and platelet derived growth factor-BB (PDGF-BB) from a bECM effectively enhances neovessel formation. It is believed that VEGF promotes chemoattraction, mitogenesis, and differentiation of endothelial cells and that PDGF enhances smooth muscle cell development for neovessels.

Using the methods and apparatus disclosed herein, a bECM may be constructed that delivers an angiogenic factor and thus fulfills several biological criteria to support wound repair. The angiogenic factor is spatially localized, protected, and delivered in a controllable and predictable manner by the bECM.

Angiogenic factors such as VEGF, FGF-2 and PDGF are typically delivered endogenously in soluble forms. Therefore, unless such factors are tethered to the bECM, pharmacokinetics will not be sufficiently controlled for predictable angiogenesis and subsequent tissue repair. Moreover, both diffusion and convective flow at the wound implant site could ‘wash out’ and dilute the local concentration gradient. Increasing the administered doses could mitigate such effects but would be problematic due to potential systemic side-effects.

In addition to the rate and amount of angiogenesis, the quality and topology of the neovascular network are critical. Delivered angiogenic molecules and ECs have been implicated as etiologic agents of vascular pathologies, including hemangiomas and other unusual vascular structures. The bECM/FGF-2 developed in accordance with the methods, compositions and apparatus disclosed herein provide an organized functional platform for normal vessel formation. The SFF ink-jet printing of bECM/FGF-2 provides a controlled patterned gradient of FGF-2 throughout the bECM. Therefore, neovessel formation is directed and organized.

Bone Tissue Engineering. In an exemplary embodiment, the methods, compositions and apparatus disclosed herein may be used in bone tissue engineering. Since angiogenesis and osteogenesis are linked, there is a strong correlation between recipient site vascularity and bone graft viability. Recent studies with knockout mice for VEGF underscore the interrelationship between angiogenesis and bone. The initial phase of bone graft healing includes chemotactic and chemokinetic signals (e.g., VEGF, PDGF, FGF-2) directing angiogenesis within the fibrin clot. Moreover, spatial and temporal patterns of GFs required for angiogenesis and osteogenesis also are required to regulate mitogenesis, cell shape, movement differentiation, protein secretion, and apoptosis.

The relatively predictable and organized set of cellular and molecular events during bone regeneration provide a mechanism for creating a controlled spatial gradient of an angiogenic factor in the bECM for bone tissue engineering. For example, when a bone fracture occurs, local blood vessels at the site are disrupted and the wound and immediately surrounding area become avascular, causing localized hypoxia and acidosis. Resident ECs respond to the hypoxic and acidotic environment and secrete VEGF and FGF. A localized and spatial concentration gradient of these angiogenic factors is produced throughout the fibrin clot, leading to an organized neovascular response antecedent to osteogenesis. Therefore, a bECM comprising an FGF-2 gradient will provide fundamental biologic responses at the wound site.

In various embodiments, a fibrin-based bECM may include two or more angiogenic molecules, including, for example, FGF-2 and PDGF (FIG. 6). Such bECMs comprising FGF-2 and PDGF are useful, for example, to regenerate healing of critical-sized defects (CSD). In certain embodiments, a tissue engineered design of a calvarial CSD has a gradient that increases from the bottom to the top of the structure. When such structure is placed into a CSD defect, the gradient encourages migration of cells in an upward direction toward the region having a higher growth factor concentration. The temporal migration of cells could also be controlled using a decreasing porosity gradient from the bottom to the top (e.g., the top is less porous than the bottom). As the cells encounter the higher density/lower porosity area of the scaffold, their migration will be slowed. In certain instances it may be desirable to print a thick or non-porous layer in one or more areas of the scaffold to prevent cell migration in a certain direction.

In other embodiments, a tissue engineered design of a calvarial CSD has a gradient of immobilized FGF-2, with concentrations higher in the center of the bECM, gradually decreasing from the center to the periphery to optimize chemoattractant and mitogenic effects that guide controlled neovessel formation. The PDGF at the center of the bECM promotes recruitment of smooth muscle cells to stabilize the neovessels. Thus, temporal control may be achieved through a spatial arrangement of PDGF and FGF-2. Furthermore, spatial variations of fibrin porosity also modulate temporal patterns. The fibrin microstructure determines the torturosity of the 3-D matrix, and manipulation of torturosity affects the bECM mechanical properties, the rate of invading cell migration, proteolysis, and growth factor availability. An increase in the fibrin compliance promotes EC differentiation in vitro.

The concentration range, direction and shape for the gradient design may be determined by the biological properties of the wound. CSD studies have reported a significant quantitative difference in osteogenic cell sources for peripheral bone, dural and subcutaneous cell sources.

Prototypic proangiogenic agents are the VEGF and FGF families. VEGF is a powerful regulator for angiogenesis, and regulating vasodilation, vessel permeabilization, and vascularogeneis. Transforming growth factor-beta (TGF-β), tumor necrosis factor-alpha (TNF-α), PDGFs, and insulin-like growth are additional proangiogenic clans. In an exemplary embodiment, FGF-2 may be used because it is angiogenic and osteogenic.

FGFs, a growing family of over nine members, are mitogenic polypeptides implicated in embryonic development, angiogenesis, regeneration, and wound healing. In various embodiments, acidic and basic FGFs, FGF-1 and FGF-2 are used for therapeutic applications for angiogenesis and bone formation. Moreover, these isoforms instigate a vasodilatory effect, mediated perhaps by an intracellular calcium-nitric oxide loop. This beneficial hemodynamic effect as well as the angiogenic capacity of FGFs merit enthusiasm as an angiogenic factor for a tissue engineered therapy. In certain embodiments, FGF-2 may be used for the positive affects of FGF-2 on bone formation and fracture healing.

Microencapsulation of biological factors by degradable polymer microspheres is a popular approach in tissue engineering. Accordingly, in certain exemplary embodiments, microencapsulation may be used to control the release of diffusible molecules over time, producing a transient diffusion gradient to regulate cell response. In other embodiments, FGF-2 may be immobilized with tissue transglutaminase (tTG). Specific binding of the FGF-2 to the bECM (i.e., FGF-2 in the solid-phase) provides maintenance of spatial patterns. Many GFs sustain residence in native ECMs through specific binding patterns. The methods disclosed herein provide bulk fabrication techniques to permit spatial patterning. The binding interactions determine GF availability and influence receptor binding, and therefore significantly impact cell responses.

The in situ printing processes disclosed herein utilize matrix materials that form porous structures without the aid of sacrificial porogens used in other SFF processes. In an exemplary embodiment, hydrogels may be used to form the structural scaffold. Suitable hydrogels include, for example, fibrin, chitosan, Collagen, alginate, poly(N-isopropylacrylamide), and hyaluronate, which can be deposited and gelled with the aid of a second component that modulates cross-linking, pH, ionic concentration, or by photopolymerization or temperature increase with body contact. In an exemplary embodiment, fibrin may be used. During wound healing, fibrin provides a foundational substratum for wound healing and angiogenesis. Fibrin results when circulating plasma fibrinogen becomes localized in a wound and following a cascade of coagulation events is finally proteolytically cleaved by thrombin and self-assembles into an insoluble fibrin network. Following this gelation event, the interconnecting fibrin fibers become stabilized by interfibril cross-linking catalyzed by transglutaminase Factor XIII (FXI II). From the plasma and platelet degranulation, a range of GFs, cell attachment molecules, proteases, and blood cell components become immobilized and entrapped within the fibrin matrix. Fibrin properties can be controlled for degradation rate and porosity. In addition, a fibrin bECM can be modified with GFs, osteoconductive bioceramics, and plasmids, so as to expand clinical versatility. Fibrin is known to bind with high affinity to FGF-2. Fibrin has demonstrated excellent biocompatibility in clinical applications. In other embodiments, other hydrogels or composites of these hydrogels may be used.

Fabrication. The exemplary bECM design illustrated in FIG. 6 provides one example out of virtually endless potential structures that may be created in accordance with the methods and apparatus disclosed herein, thereby providing versatile and new opportunities for tissue engineers. The methods, compositions, and apparatus disclosed herein provide the capability to fabricate bECM/GF designs with spatial patterns and to concurrently position a complex biological therapy into a patient using solid free-form fabrication (SFF).

SFF refers to computer-aided-design/computer-aided-manufacturing (CAD/CAM) methods that can fabricate automatically, complex shapes directly from CAD models. SFF processes are based on a layered manufacturing paradigm that builds shapes by incremental material deposition and fusion of thin cross-sectional layers. While SFF processes are used predominantly for industrial applications, SFF may also be used to manufacture bECMs with controlled microstructures for tissue engineering applications. Therapeutic factors can be added to biomaterial structures as they are built with SFF to precisely control the 3-D spatial distributions of the factors throughout these structures. Others have reported SFF based on photo-activated biological hydrogels with proteins and fibrin “bioplotters”, however neither approach addresses spatial control of GFs. In certain exemplary embodiments, a SFF system, such as the system illustrated in FIG. 2, may be employed to engineer a bECM based on fibrin, or other native ECM materials, with spatial distributions of GFs. In certain exemplary embodiments, SFF processes will be utilized to manufacture fibrin-based bECMs with concentration gradients of GFs. The methods and apparatus disclosed herein overcome problems with surgical implantation of certain fragile bECMs by making the SFF process compatible with in situ deposition of the bECM/GFs directly into the wound site (FIG. 7). In an exemplary embodiment, focused ink-jet print heads are used to co-deposit fibrinogen, thrombin, FGF-2, and cross-linking factors to produce, layer-by-layer, by local mixing of the droplets at the printed surface, a 3-D patterned bECM/FGF-2 structure.

Printing in situ. In situ fabrication of bECMs, by e.g., printing, is useful for a variety of biological and clinical applications. In situ fabrication may useful to prevent damage to a bECM during surgical handling and may avoid difficulties in accurately matching the prefabricated bECM dimensions to a specific defect geometry.

An exemplary in situ apparatus with a miniaturized ink-jet printer is shown in FIG. 7. The device may be registered to the patient with a stereotactic device that will deposit bECMs/GFs directly at a desired location. In exemplary embodiments, fibrin-based bECMs with concentration gradients of FGF-2 are printed in situ.

The methods, compositions and apparatus disclosed herein may be used for a variety of applications, including, for example, regeneration of epithelial gastrointestinal mucosa and articular cartilage. Moreover, skin analogues could be printed, e.g., ‘ink-jetted’, onto burns. SFF spatial gradient inkjet technology will enable tissue engineering therapies to meet clinical challenges through controlled 3-D pattern deposition of biological materials, and direct in situ deposition of tissue engineering constructs into a recipient site.

### Example 1

SFF Production Offibrin bECMs

In one embodiment, individual focused ink-jet print heads may be used to co-deposit fibrinogen, thrombin, FGF-2, tTG, and buffer to produce a bECM-fibrin matrix with specified 3-D spatial patterns of FGF-2 and microstructure. The bECM is fabricated layer-by-layer by local mixing of the droplets at the printed surface to produce the structure. The SFF apparatus used microdispensing solenoid valves (manufactured by The Lee Company, Westbrook, Conn.), which can produce droplets as small as 10 nanoliters, to deposit solutions of fibrinogen, thrombin, and a surrogate growth factor (FIG. 8). Jetting devices that can print smaller droplet volumes may also be used. The dispensing devices are mounted to computer controlled X-Y stages (Parker Hannifin, Wadsworth, Ohio) that move a substrate relative to the focused dispensing devices. By varying the relative amounts of the deposited components the fibrin porosity and GF concentration throughout the 3-D space is selectively controlled. The apparatus may be configured so that the net deposition volume at each point in space is held constant. For example, if the firing rate of the thrombin print head is decreased, the firing rate of the fibrinogen print head is proportionally increased. Toggling the firing rates between thrombin and fibrinogen modulates the porosity developed in the bECM.

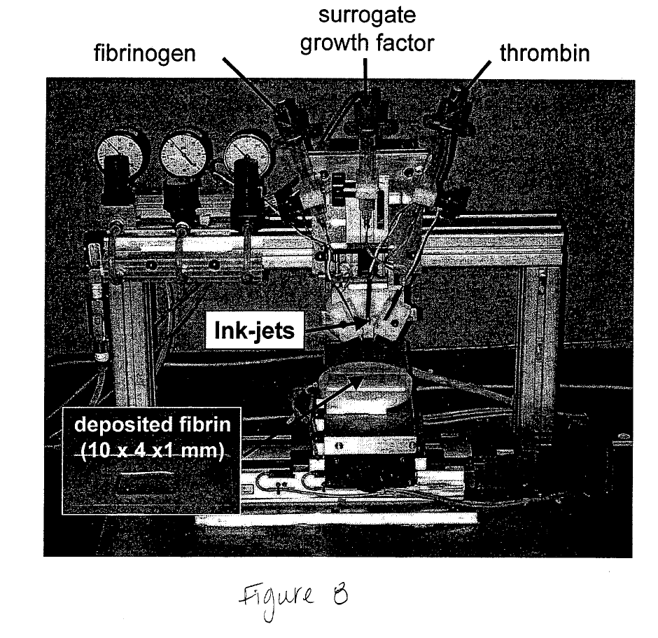

FIG. 10A shows a 1 mm thick, 4 mm×10 mm fibrin matrix with a microstructure that is native in appearance that has been produced using the methods disclosed herein. FIG. 10B shows a fibrin bECM with a gradient of Cy3 labeled dextran (10,000 MWt) as a surrogate factor. FIG. 10C shows a gradient of fibrin porosity. Fibrinogen concentrations ranging from 5 to 25 mg/ml, with thrombin held constant at 1 NIH unit/ml were fabricated by this printing process. Printing activated Cy3 alone (cross-links directly to fibrin) demonstrated persistence of printed patterns over several days at 23° C. in PBS, in contrast to reacted Cy3 (1000 MWt, does not bind to fibrin), which rapidly diffused throughout the fibrin gel, with a loss of pattern definition within 15 to 30 min. The bio-inks were deposited onto fibrin-coated glass substrates.

### Example 2

Production of an Exemplary SFF Deposition System

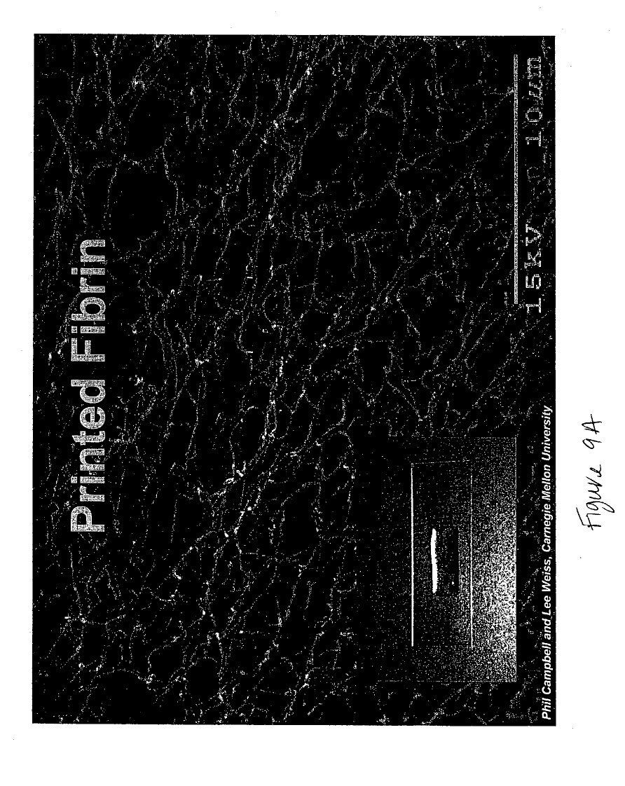

An exemplary SFF system for dispensing bio-inks is shown schematically in FIG. 11. The SFF process begins with a 3-D computer model representation of the bECM/FGF-2 therapy. The model specifies the fibrin porosity and the FGF-2 volumetric concentration at each point in 3-D space. The ACIS geometric modeling kernel is used for this representation. The bECM/GF computer model is then subdivided into discrete ‘voxel’ representations and then into layers of voxels according to the volumetric resolution of the printing system. Each voxel, or cube unit, in each layer has an associated biological composition specified by the fibrin porosity and FGF-2 concentration. A mixture planner determines the volume each bio-ink that must be deposited at each point in space to achieve the specified biological composition. The net deposition volume at each point may be held constant, e.g., if the amount of thrombin is decreased to increase porosity, then either the amount of fibrinogen or buffer, or both may be increased proportionally.

Next, the volumes of the biological factors to be deposited in a given layer are encoded as gray-level values and stored in image buffers. Separate image buffers are used for each biological factor. The image buffers input data into one or more processors programmed to control the operation of the ink jets. As the stages move, signals from the motor encoders are fed back to the processor(s) to synchronize firing of the ink-jets with the table motion. The net volume of liquid deposited at each location is dependent upon the droplet volumes and the number of droplets deposited. The droplet volume is dependent upon numerous physical parameters, such as nozzle diameter and ink viscosity, but can also be adjusted by the modulating the waveform driving each print head.

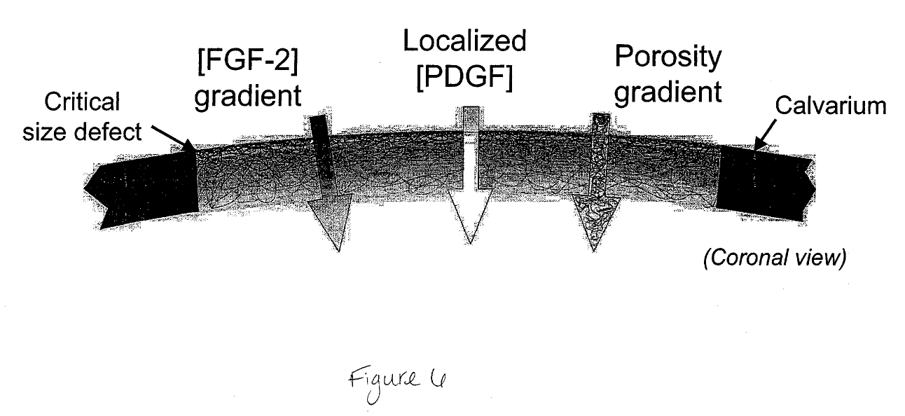

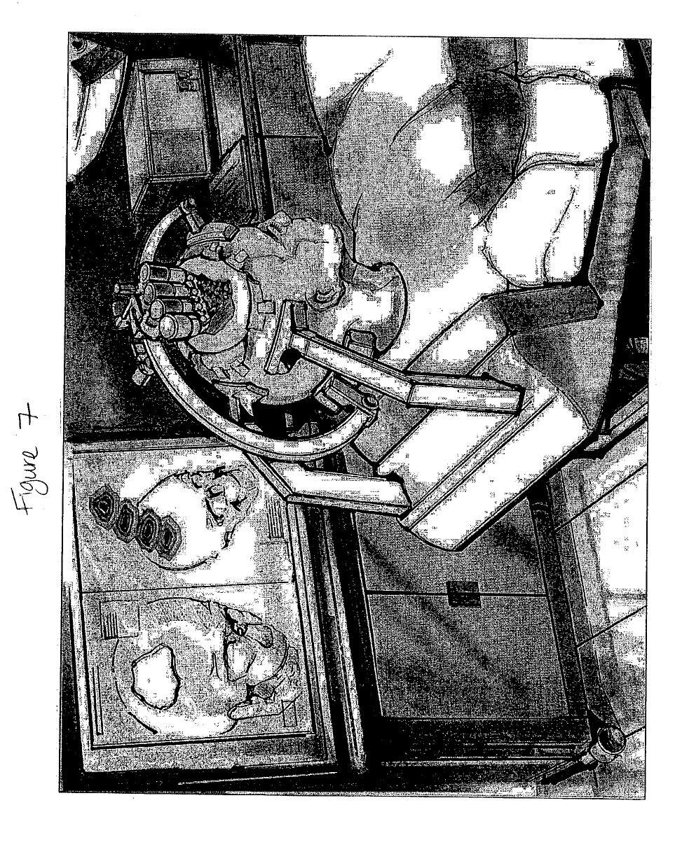

The dispensing devices includes drop-on-demand (DOD) piezoelectric inkjet print heads (PIJPs), manufactured by Microfab, Inc. (Plano, Tex.), which can produce droplets as small as 30 picoliters. The PIJPs are used for depositing, high-resolution FGF-2 gradients and precise amounts of thrombin, tTG, and buffer. Micro-dispensing solenoid valves may be used (FIG. 9) to deposit the higher viscosity fibrinogen inks, but at a lower resolution. A precision syringe pump may be added in series with this valve to increase the viscosity capability, as well as the printing resolution to approximately I nanoliter. The ability to print the lower viscosity FGF-2, tTG, or thrombin inks at higher resolutions will not be affected. The dispensing devices are mounted to computer controlled X-Y stages (Parker Hannifin, Wadsworth, Ohio) that move the substrate (i.e., slide, animal, etc.) relative to the print heads. The Z-axis is manually adjusted to set the substrate-to-printhead standoff height. A servo-controlled Z-axis may also be used. Heaters may be built into the ink reservoirs and print heads, and a spot infrared heat source may be focused on the target to ensure consistent deposition performance and control gelation rate.

A deposition strategy that includes the sequence in which voxels are deposited and the timing between depositions of voxels is specified. For example, one deposition strategy may be to first deposit every other voxel in a layer, and then make a second pass to fill in the other voxels. This would allow sufficient time for the fibrinogen to gel in each location, thus reducing ‘bleeding’ between adjacent voxels. Another deposition strategy may include depositing bio-ink in a circular pattern formed by, for example, a series of circular deposition passes. After a set of strategies is specified, the motion planner sets the raster trajectory parameters for of the linear stages.

### Example 3

Synthesis of Bio-inks

Gelation rate, structure, and material properties of fibrin gels are determined by relative concentrations of fibrinogen and thrombin, pH, ionic strength and other biophysical parameters present during fibrin polymerization. For example, fibrinogen concentration directly affects fibrin gel strength as does cross-linking of the fibrin gel with FXIII which also protects fibrin from plasmin proteolysis thus modulating bECM degradation. The resulting 3-D microstructural properties of the fibrin gel play a decisive role in EC migration, proliferation and angiomorphogenesis. Typically, FGF-2 and VEGF stimulation of migration is enhanced by more rigid or less porous fibrin gels, whereas capillary morphogenesis is enhanced by less rigid or more porous gels.

The bio-inks disclosed herein permit differential control of fibrin variables at the micro-scale during fibrin gelation. In an exemplary embodiment, fibrinogen, thrombin, FGF-2, tissue transglutaminase (tTG), and dilutant buffers are printed. For all bio-inks, pH and ionic strength are held constant in 100 mM Tris buffer, pH 7.0, containing 150 mM NaCl and 5 mM CaCl. Structural bio-ink components in their simplest form consist of fibrinogen and thrombin. These two components form the base for both a native thrombus formation and commercial fibrin glue. The addition of TGs cross-links fibrin fibrils and stabilizes the fibrin polymer, thereby improving mechanical properties. TGs are Ca²⁺-dependent enzymes that catalyze post-translational modification of proteins through the formation of γ-glutamyl-ε-lysine cross-links between polypeptide chains. Plasma FXIII is activated by thrombin and is primarily associated with the covalent cross-linking of fibrin fibrils. A stronger clot is produced with FXIII. tTG is widely distributed in cells and tissues and does not require proteolytic activation. TGs impart fibrolytic resistance by cross-linking α2-antiplasmin to fibrin fibrils and by cross-linking the fibrin α-and/or γ-chains. TGs have a broad range of substrate proteins including fibrinogen/fibrin, fibronectin, plasminogen activator inhibitor-2, α2-antiplasmin, IGF binding protein-1, osteonectin, β-casein, collagen, laminin, and vitronectin. There is differential substrate specificity between TGs. tTG is preferred because it does not require thrombin activation, is readily available, and because it is a factor in osteogenesis.

Human plasminogen-free fibrinogen and human thrombin may be purchased from Enzyme Systems Research Laboratories (South Bend, Ind.), tTG from Sigma (St Louis, Mo.), and human recombinant FGF-2 from ReproTech, Inc. (Rocky Hill, N.J.). Such materials are also available from GMP facilities and FDA approved sources. Fibrinogen is printed at concentrations in the range of 4-75 mg/ml. Four mg/ml is the concentration of native fibrin clots, and up to 130 mg/ml is used in commercially available fibrin glue formulations such as Tisseel. Thrombin concentrations between 1 to 50 NIH units/ml will be tested to modify gelation time, fibrin fibrillar diameter and porosity. FGF-2 bio-inks will consist of FGF-2 concentrations between 1-12 ng/ml.

Temperature plays an important role in stability of bio-ink protein components and the rate of fibrin gelation. Ink temperature is maintained in the reservoirs and print heads at 23° C. All protein-based ink components are stored at -70° C. or freeze-dried prior to printing to maintain viability.

There are three primary factors to consider in formulating the inks—stability, jetdroplet control, and mixing. Ensuring stability of the inks requires avoiding degradation of the biological components, an issue dealt with through care in sterilization and temperature control.

The resolution of the structures formed depends on the ability to control the delivery rate and dimensions of the droplets formed during jetting. Droplet formation depends on physical parameters of the fluid, viscosity (μ), surface tension (σ) and density (ρ), and the parameters of the ink-jetting including drive-waveform, nozzle radius (R) and average velocity of the droplets (V). In the case that the fluids are essentially Newtonian, formation of the droplets is dictated by two dimensionless groups:

Re=2ρV R/μ Oh²=μ²/(2ρσR)

The Reynolds number (Re) quantifies the relationship between inertial forces and viscous forces—it indicates whether the flow in the nozzle is laminar or turbulent. The Ohnesorge number (Oh) characterizes the relative strength of viscous forces to interfacial forces. The magnitudes of Re and Oh define the drop size. Since the jet is driven by a forced disturbance, the influence of initial disturbance amplitude and wavelength are considered. Rheology is determined with standard rheometric techniques including rotational rheometry and capillary viscometry. Interfacial properties including static and dynamic surface tensions are determined using techniques such as DuNouy ring and bubble tensiometry. These methods may be used to define the process parameters of the jetting to avoid regions of gross jet instability, spurting or satellite drop formation.

The assumption that the fluids are Newtonian is reasonable. However, droplet formation is strongly influenced by even the slightest elasticity in a fluid. Viscoelasticity may be investigated through rheometric studies. Changing of the formulation and/or alteration of the process parameters may be utilized to deal with issues of die swell and viscoelastic jet formation.

Once delivered to the printed surface, the components interact to form a homogeneous material at the point of impact. Modeling of this type of multicomponent gelation/diffusion/mixing problem is complex, but the framework for simple qualitative modeling exists within the field of transport phenomenon and reactor engineering. Gelation kinetics may be characterized in the bulk by measuring the elastic modulus (G′) as a function of time. Results may be compared to previous work on gelation of other biopolymers (e.g., collagen) and synthetic polymers to develop simple models for gelation. Bulk measurements of gelation is problematic for stiff gels due to issues of linearity, slip and fracture. However, this method provides accurate measurements of modulus as a function of reaction time for the initial stages of the cross-linking. Since dispersion in the composite prior to complete gelation is desired, modulus is the relevant physical property. Modeling of mixing assumes 1- and 2-dimensional mixing, Newtonian fluid mechanics, and simple diffusion and convection arising from droplet spreading. The viscosity increase with reaction may also be included and dimensionality increased. Model viability may be verified by comparison to experiments performed on relevant model systems (i.e., no added catalyst to investigate mixing without gelation).

Models that assume a stagnant drop delivered to the surface may be enhanced with information about drop impact and dynamic spreading that may be obtained using high-speed video capture.

Calibrate and tune system. The exemplary SFF system can spatially control two bECM variables (β): fibrin porosity (ρfibrin) and FGF-2 concentration (C_{FGF-2}), or

β=[ρfibrin, C_{FGF-2}]

is a complex function of dozens of printing and ink parameters (w) including, for example, bio-ink concentrations, ink rheology (viscosity, surface tension), ink jet printing (IJP) waveforms (rise and fall times, dwell, amplitude, frequency), motion trajectories (speed, printer to substrate distance), deposition strategies (line spacing, droplet timing), nozzle diameter, and temperature.

Regression models are first established, for each ink formulation, to determine droplet diameter (D_{drop}) and velocity (V_{drop}) as a function of the waveform parameters. Diameter and velocity may be measured using video imaging with stroboscopic lighting. The smallest droplet size (D_{drop-min}), minimal printer-to-substrate stand-off height (Hₘᵢₙ), and minimum droplet velocity (V_{drop-min}) that produces repeatable droplet coalescence and mixing at the substrate surface, which is dependent on the accuracy and repeatability of focusing the droplets at the substrate, are determined. Droplets may deviate from nominal targeted locations due to small variations in the relative height of the growing fibrin substrate and due to random wetting variations at the nozzle tip. For each ink concentration of biological factors (C_{*factor}), a regression model is established, h, or a look-up-table that relates β to the net deposited volume of each factor at D_{drop-min}, Hₘᵢₙ, V_{drop-min}:

B=h(VOL_{fibrinogen}, VOL_{thrombin}, VOL_{transglutaminase}, VOL_{FGF-2}, VOL_{Buffer})

subject to the constraints: 5

$${\sum }_{i = 1}^{} {VOL}_{i} = equal a constant ( voxel size ) , and fixed {C}_{factor}^{*}$$

The voxel resolution is a function of Ddrop. The mixing planer uses these models to set the volumes to be jetted.

Validate printed bECMs. bECMs will be printed on Millicell polycarbonate membrane-based culture plate inserts (Fisher, Pittsburgh, Pa.). Prior to printing, both sides of the membrane will be treated with 4 mg/ml fibrinogen solution in 200 mM sodium carbonate buffer, pH 9 overnight at 4° C. Fibrinogen films will be air-dried and inserts stored at 4° C. until printing. The printed fibrin and FGF-2 patterns will be validated using SEM and fluorescent microscopy. The persistence of FGF-2 patterns will be validated using fluorescence and ¹²⁵I-FGF-2 labeling. For each design, C_{FGF-2} and pfibrin will be measured throughout the bECM at the voxel resolution of the design model. A computer model of the deposited bECM/FGF-2 structure, β_{measured}(x,y,z), will then be established using this data. Six replicates of each design will be measured. The regression and design model parameters will be compared to assess the accuracy and repeatability of the SFF system.

SEM. Printed bECMs will be fixed with 2.5% gluteraldehyde in PBS, pH 7.4 at 4° C. for at least 24 hr. Gels will be dehydrated in increasing series of ETOH to 100% followed by critical point drying using CO₂ (Pelco CPD2 Critrical Point Drier). Samples will be mounted on SEM sample stubs and sputter coated with gold-palladium (Pelco SC6 Sputter Coater). Samples will be examined in a Hitachi 2460 scanning electron microscope and Quartz PCI imaging system software.

Fluorescence confocal laser microscopy. Fibrinogen bio-inks will be augmented with Cy5 labeled fibrinogen (5% vol:vol to unlabelled fibrinogen). FGF-2 will be augmented with Cy3 labeled FGF-2 (5% vol:vol to unlabelled FGF-2). Prelabeling will permit fluorescent identification of printed patterns. Printed bECMs will be fixed and confocal microscopy performed using a Zeiss confocal LSM1 0 microscope equipped with 5 mW AR 488/514 nm and a 5 mW HE/NE 633 nm lasers. A Zeiss Plan-Neofluar 20×0.5 NA water immersion objective will image sections in 1 μm, or better, increments. Images will be processed using Zeiss LSM software.

Persistence of FGF-2 p rinted patterns. Printed patterns will be immediately fixed or placed in excess phosphate buffered saline, pH 7.4 (PBS), containing 0.02% sodium azide for various times (0, 0.5, 1, 4, 8, 24, 72 hrs) at 23° C. using time 0 as the control. For selected experiments, we will substitute ¹²⁵I-FGF-2.

Determination of FGF-2 biological activity. Selected bECM designs will be printed on 12 mm glass coverslips. Printed bECMs will be placed in 24 well tissue culture plates for ³H-thymidine assay. Human umbilical ECs (HUVECs) will be purchased from Clonetics (BioWhittaker, Inc., Walkersville, Md.) and maintained according to supplier's instructions. Cells will be grown to ˜70% confluence. Cells will be seeded onto bECM at 20,000 cells/well in serum-free media. After 48 hr culture, 0.5 μCi ³H-thymidine will be added to the wells. After overnight culture, bECMs will be trypsinized to dissolve fibrin matrix and cells will be washed with PBS and ³H-thymidine incorporation determined by standard protocol.

Statistical Analysis. Quantitative data will be analyzed by multiple analysis of variance (ANOVA) and Tukey's post-hoc test for multiple comparison analysis. The level of significance will be p<0.05.

Since the stiffness of the fibrin matrix decreases with fibrinogen concentration, slumping may become a problem at lower fibrinogen concentrations. Varying the pH and ionic concentrations alter mechanical properties while maintaining fibrinogen concentration. Alternatively, lateral support for bECMs can be provided using plastic rings glued to the printed surface. Ring dimensions will be equivalent to the bECM.

tTG crosslinking of FGF-2. A broad range of substrate proteins for FXIII and tTG have been identified, including fibrinogen/fibrin, fibronectin, plasminogen activator inhibitor-2, α2-antiplasmin, IGF binding protein-1, osteonectin, Pcasein, collagen, laminin, and vitronectin. To account for differences in substrate specificity, different TGs or FXIII may be used. Alternatively, FGF-2 may be cross-linked to a dilute solution of fibrinogen prior to formulation of the FGF-2 bio-ink. FGF-2 specifically binds fibrinogen via standard reaction using BS³ (a water soluble bis(sulfosuccinimidyl) suberate) from Pierce (Rockford, Ill.). This cross-linker may be used to immobilize IGF-I to metal surfaces and it is biocompatible. Should bECMs require higher FGF-2 concentrations, an oligoglutamine moiety may be coupled onto FGF-2 via BS3. Furthermore, the exact nature of the binding region can be tailored to maximize its reactivity; for example, chain length and composition can be altered. Various oligopeptides can be synthesized which are rich in both glutamine and the facilitating amino acids. Crosslinking heparin to fibrinogen or fusion peptides using TG substrate sequences may be utilized. Engineered peptides, fusion proteins, and other such molecules may also be used to promote attachment of therapeutic agents such as drugs, growth factors, etc. to matrix components either directly as a fusion protein (i.e., a growth factor with a TG substrate component with out without a protease cleavage site) or an engineered peptide (i.e., such as a heparin binding domain sequence with a TG substrate sequence that may be used to immobilize heparin to serve as a generic binder for proteins containing heparin binding domains).

### Example 4

Evaluation of Angiogenesis of bECM Designs with 3-D Spatial Concentration Gradients

bECM Designs and Fabrication. A range of bECM/FGF-2 designs, which are depicted in FIG. 12, were selected: 1) A solid-phase concentration gradient of FGF-2 will promote a controlled angiogenic response; and 2) concentration patterning of solid-phase FGF-2 within a fibrin-based bECM will result in an improved angiogenic response in comparison to designs based on uniform solid-phase distributions of FGF-2. These designs will be fabricated using the ink-jet deposition system described above.

There are three design sets representing unidirectional (FIG. 12C), uniform (FIG. 12D), and radial (FIG. 12E), distributions of FGF-2, and a control without FGF-2 (FIG. 12F). Each design has a uniform distribution of fibrin porosity. In FIGS. 12A-F, C_{FGF-2} is the specified volumetric concentration of printed FGF-2 and ρ_{porosity} is the specified fibrin porosity. M_{FGF-2} is the magnitude of the FGF-2 pattern designs and M_{fibrin} is the specified value of porosity while C*_{FGF-2} and C*_{fibringen} are the bio-ink concentrations. The correlation factors relating C* to M, which are required by the mixing planner, will be determined as described herein.

The unidirectional and radial gradients are specified with a linear decay. These shapes are merely exemplary. For example, non-linear gradients are also contemplated. Furthermore, the attenuation of FGF-2 concentration to 10% of MFGF-2, is also merely exemplary of concentration suitable for stimulating migration at the cell/bECM interface.

Each design will be fabricated as discs (8 mm diameter by 2 mm thick) (FIG. 12A). The substrates to be printed onto are described below. Changing the fibrinogen concentration while keeping thrombin fixed at 1 NIH unit/ml will modulate the fibrin porosity. Three levels of fibrin porosity, printed as uniform distributions, using fibrinogen bio-ink concentrations of 4, 10 and 25 mg/ml will provide a range of fibrin porosity to influence migration. Two levels of FGF-2 concentration magnitudes will be tested based on bio-ink concentrations of 10 and 25 ng/ml for the in vitro studies, and 1 and 5 ng/ml for the CAM studies. These concentration ranges are reported to stimulate endothelial cells and angiogenesis in CAM.

Following the fabrication, replicates will be used for in vitro or CAM assays immediately or placed in serum-free media containing 50 μg/ml BSA (Insulin RIA grade, Sigma, St. Louis, Mo.) and 1 μg/ml aprotinin at 23° C. These bECM samples will be incubated with media changes for optimum time to remove unbound FGF-2. Holding the temperature at 23° C. and the addition of the protease inhibitor, aprotinin, will stabilize the fibrin structure.

In Vitro Evaluation. The effectiveness of tissue-engineered constructs is often evaluated in vitro prior to assessment in vivo. In vitro results may not directly translate to in vivo results. However, compared to in vivo experimentation, in vitro experimentation is associated with reduced expense, increased experimental turnover rates, and more selective control of associated variables. These considerations support in vitro experimentation in the tissue engineering design process.

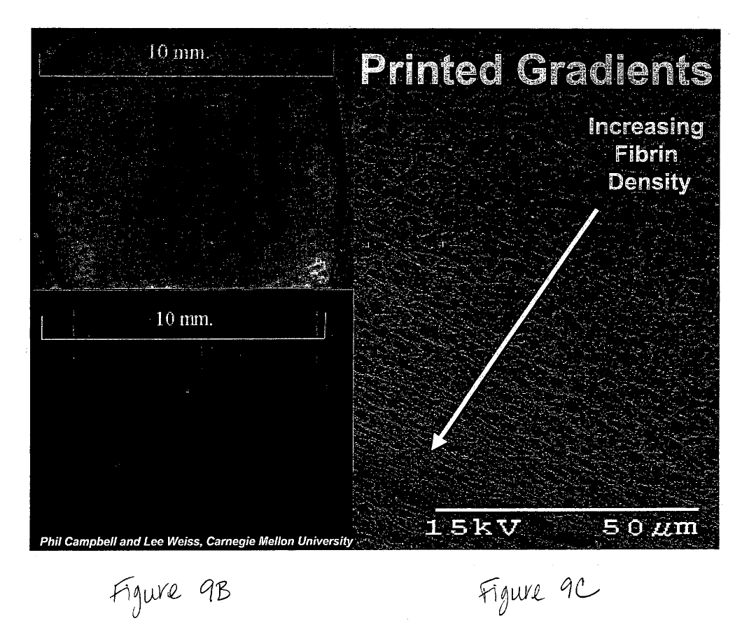

In vitro studies may be used to examine directed cell migration and proliferation of ECs in response to bECM/FGF-2. Millicell polycarbonate membrane-based culture plate inserts (Fisher, Pittsburgh, Pa.) will be utilized as a printing substrate (FIG. 13A). The fibrin/fibrinogen readily adsorbs to these tissue culture treated membranes; thus anchoring the printed structures. The 12 μm pore size will provide unimpeded cell migration across the membranes. All procedures will be performed under sterile conditions. Prior to printing, both sides of the membrane will be treated with 4 mg/ml fibrinogen solution in 200 mM sodium carbonate buffer, pH 9 overnight at 4° C. Fibrinogen films will be air-dried and inserts stored at 4° C. until printing. Coated culture plates will be inverted onto a Teflon mandrel prior to printing to insure that jetted liquids do not pass through the porous membrane prior to gelation. Once the bECM designs are printed, inserts will be inverted and placed into 24-well tissue culture plates (FIG. 13B). 8 replicates of each design will be printed and controlled for both migration and proliferation experiments. Human umbilical endothelial cells (HUVECs) will be purchased from Clonetics (BioWhittaker, Inc., Walkersville, Md.) and maintained according to supplier's instructions. Cells will be grown to ˜70% confluence in 100 mm culture dishes, labeled with 50 μCi ³H-thymidine overnight. Labeled cells will be trypsinized and seeded into insert wells to ˜80% confluence. After 24 hr, inserts will be removed from culture and the bECMs removed using a razor blade, placed into scintillation vials containing 0.5 ml 0.5 N NaOH. After 1 hr, 37° C., solubilized samples will be counted for radioactivity. Based on persistence studies as described herein, selected bECM samples will be placed directly in assay following printing or held in buffer +100 ng/ml aprotinin for indicated time points to maximize removal of unbound FGF-2.

To test cell proliferation in the bECM, unlabelled HUVEC cells will be seeded over printed bECM samples similar to the method used in the migration studies. 0.5 μCi will be added per sample at 48 hrs post-seeding. After 24 hr, the bECM will be scraped from the insert membrane and transferred to sample vials. Samples will be prepared for scintillation counting by standard protocols.

For selected experiments, migration and proliferation experiments will be performed without ³H-thymidine labeling. After 24 hr for migration studies and 72 hr for cell proliferation studies, inserts will be removed and fixed with 4% paraformaldehyde, cells permeabilized with 0.1% triton-X 100. Cell nuclei will be stained using DAPI to identify cells within bECM. Fibrin matrices will be stained using cy-5 as described in herein. Quantity and distribution of cells will be determined by confocal microscopy.

CAM Evaluation. A scientifically accepted alternative to animal models is the chorioallantoic membrane (CAM) model. The CAM is a vascular extraembryonic membrane located between the embryo and the eggshell of developing chicken egg. Angiogenesis and the CAM have become an important in vivo biological assay to screen therapies for wound repair and blood vessel development.

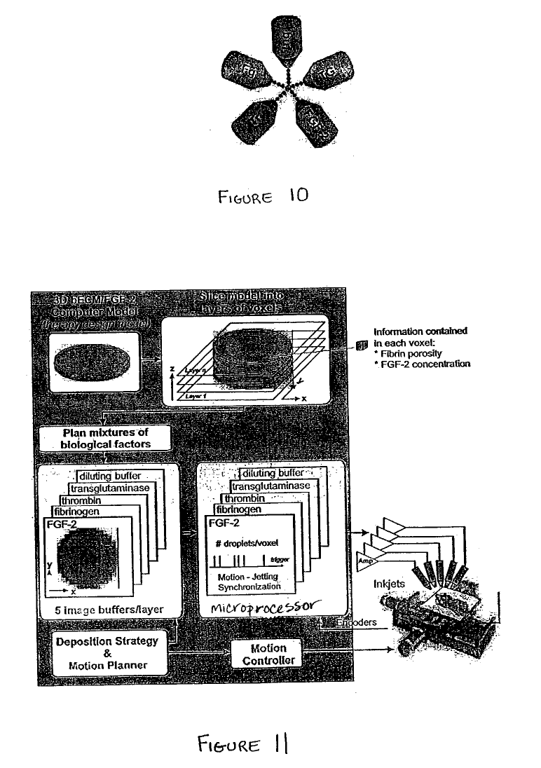

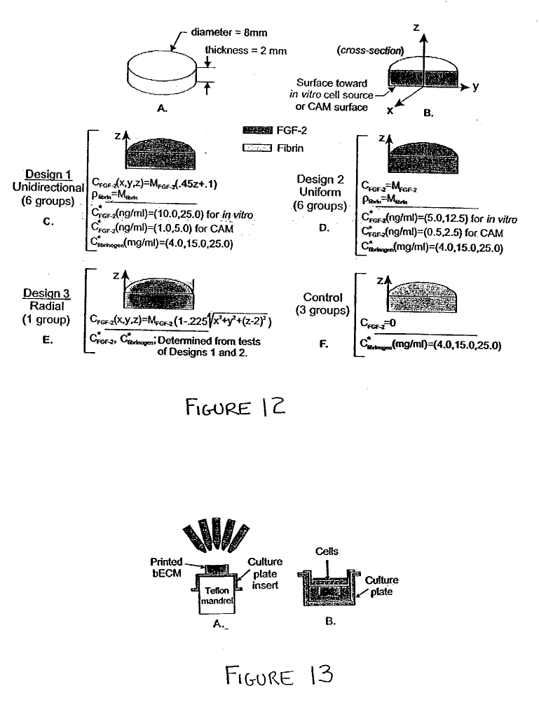

CAM will be used to assess angiogenesis in response to the fibrin bECM/FGF-2 designs. To ensure bECM fixation to the CAM a cutting device has been constructed to make a 17 mm diameter hole in the horizontal center of eggs (FIG. 14A). An optically clear plastic insert (15 mm OD×10 mm ID) was developed to create windows for focused treatment application and subsequent in situ assessment (FIG. 14B). Placing sample constructs of smaller size than the insert provides a border region surrounding the construct within the viewing window allows in situ observation of the directed vascular ingrowth (FIG. 14C).

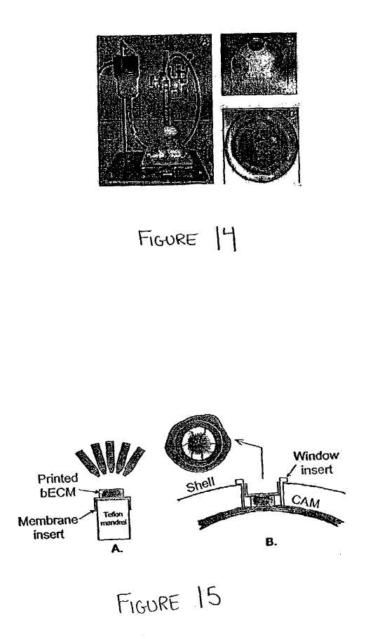

CAM Assay. The CAM assay consists of incubating fertilized White Leghorn eggs at 37.8° C. in 60% relative humidity. On day three, eggs are opened using a mid-horizontal orientation in the cutting device (FIG. 14A). Removal of 0.5 ml of albumin from the large end of the egg prior to cutting drops the embryo from the cutting site, protecting it from vibration and surgical trauma. Porous medical tape placed over the hole minimizes evaporative loss and prevents contamination. On day 8, window inserts are placed through the hole and rest directly on the CAM (FIG. 15B).

The printed bECM/FGF-2 will be placed on the CAM on day 10 (FIG. 15B). In situ imagining will be digitally recorded for image processing from days one through eight post bECM/FGF-2 application. The bECM/FGF-2 therapy placed into the CAM inserts will be recovered at this time and prepared for histological analyses of angiogenesis. Embryos, membranes, and bECM will be fixed in ovo in Bouin's fluid. The window/CAM area will then be removed, dehydrated and embedded in paraffin. Serial sections of 8 tam will be made in a plane parallel to the CAM surface. Sections will be stained using 0.5% toluidene blue. Angiogenesis will be evaluated with a Zeiss Axiophot microscope interfaced with an image analysis system using Zeiss imaging software.

Statistical analysis. Quantitative data will be analyzed by multiple analysis of variance (ANOVA) and Tukey's post-hoc test for multiple comparison analysis. The level of significance will be p<0.05.

If the printed solid-Phase FGF-2 does not extend through the membrane pores to directly contact in vitro seeded Ecs, random migration across the membrane may not result in sufficient numbers of ECs initially contacting the printed FGF-2 patterns. In this case, additional print fluid-phase FGF-2 at the bECM may be to interface with the membrane.

### Example 5

Examination of in situ bECM Fabrication in a Rat Calvarial Defect

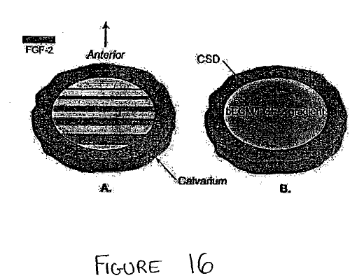

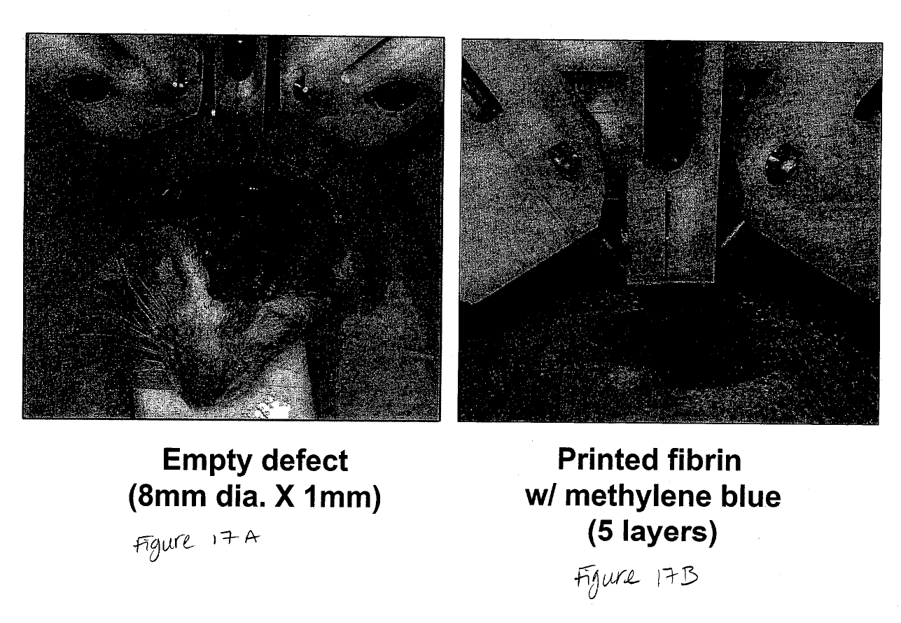

Fabrication of in situ bECM In situ printing of a fibrin bECM/FGF-2 into a wound may be examined using a rat calvarial defect. A total of 24 rats will be used, 12 rats per printed bECM/FGF-2 pattern. (2 patterns printed into rats directly (6 rats/pattern)=12 rats; 2 patterns printed into rats intravenously injected with Cy7-fibrinogen (6 rats/pattern=12 rats). The discrete pattern in FIG. 16A will be printed into rat CSDs to establish standards to calibrate the printed radial gradient in FIG. 16B. CSDs will be created in Sprague Dawley rats using standard protocol. Male rats, 300-350 g will be anesthetized by intramuscular injection of a combination of 75 mg/kg ketamine and 0.75 mg/kg acepromazine. After achieving an appropriate level of anesthesia, 3 ml saline will be delivered subcutaneously as a prophylactic against dehydration during surgery. The calvarial area will be shaved and depilated in the standard manner using aseptic procedures. An 8 mm diameter CSD will be prepared in the parietal bone of the calvarium with an 8 mm trephine and copious irrigation with physiologic saline. The craniotomy segment with the attached periosteum will be removed gently, leaving the dura intact. An example of an empty CSD defect in the parietal bond of a rat clavarium is shown in FIG. 17A. The CSD will be registered with our printing device using a standard rat head stereotactic device (Harvard Instruments, Boston, Mass.). A radial bECM/FGF-2 design (from Design 3, FIG. 12E) will be fabricated in situ using the printing device described herein. The 3-D spatial control in situ will be examined. Fibrinogen bio-inks will be augmented with Cy5 labeled fibrinogen (5% vol:vol to unlabelled fibrinogen). FGF-2 will be augmented with Cy3 labeled FGF-2 (5% vol:vol to unlabelled FGF-2). This pre-labeling will permit fluorescent identification of printed patterns. Post-printing, animals will be euthanized within an hour by opening the thoracic cavity. Animals will not regain consciousness. The complete calvarium will be removed and fixed using freshly prepared 2.5% gluteraldehyde in PBS at 4° C. for at least 48 hr. An example of in situ printing into rat parietal bone defect using fibrin with methylene blue is shown in FIG. 17B.

Validation of in situ printed bECMs. 3-D patterns of Cy3-FGF-2 and Cy5-fibrin will be determined on intact calvarial samples using confocal microscopy. Subsequently, samples will be equilibrated in PBS, pH 6.0 for 24 h, and then immersed in activated Cy7 in PBS pH 6.0 for 24 h. This will label all tissues a contrasting fluorescent color. The different excitation/emission wavelengths permit the co-localization of printed FGF-2 and bECM fibrin to surrounding native fibrin. This will provide evidence of the bECM/peripheral rim interface.

Following validation of the printed patterns, printing will be carried out in rats that have had Cy7 labeled fibrinogen intravenously injected to label endogenous fibrin sources. Therefore, during surgery the now “host” Cy7-fibrinogen will label the peripheral fibrin clot, while the Cy5-fibrinogen will label the printed bECM fibrin. The differential colors between these two fluorochromes will permit the examination of the bECM/wound peripheral interface.

Cy7-fibrinogen labeling the rat bloodfibrinogen pool. Rat fibrinogen pools will be labeled to ˜5% wt/wt by injecting IV 5 mg Cy7-fibrinogen via the rattail vein. This is based on the following calculations: Total blood volume in the rat is 5.6-7.1 ml/100 g body weight (BWT). An average blood volume for rats to be used in this application becomes 6.35 ×3.5 (350 g wt) or 22.225 ml. With a fibrinogen concentration of 190 mg/dl or 19 mg/ml this gives a total fibrinogen concentration of 48 mg or 5%=5 mg.

Statistical Analysis. Quantitative data will be analyzed by multiple analysis of variance (ANOVA) and Tukey's post-hoc test for multiple comparison analysis. The level of significance will be p<0.05.

Infused Cy7-fibrinogen may not produce sufficient labeling of endogenous wound fibrin in conjunction with printed bECM. Therefore, aside from altering the time from infusion to surgery or the concentration of Cy7-fibrinogen infused, immunofluorescent staining with antifibrinogen and Cy7 labeled antibodies fibrinogen may be used. This will permit the visualization of Cy7 without interference from Cy5-fibrinogen. Fibrin co-visualized for both Cy3 and Cy7 represents printed fibrinogen, while Cy7 visualized without Cy3 is native fibrinogen.

Excessive bleeding may interfere with controlled in situ printing by corrupting the specified bECM/FGF-2 pattern by dilution, convection, and interference with gelation. The surgical procedure used to produce rat calvarial CSDs does not produce excessive bleeding. However, should this problem occur a fine fibrin spray may be applied to prepare the surgery site for printing.

EQUIVALENTS

The present disclosure provides among other things methods, compositions and apparatus for creating biomimetic extracellular matrices with patterned 3-D gradients of therapeutic factors. While specific embodiments have been discussed, the above specification is illustrative and not restrictive. Many variations of the apparatus, methods, and process disclosed herein will become apparent to those skilled in the art upon review of this specification. The appended claims are not intended to claim all such embodiments and variations, and the full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: U.S. Pat. Nos.: 5,460,831; 5,738,824; 5,851,229; 6,004,573; 6,124,265; 6,143,293; 6,165,486; 6,217,894; 6,302,898; 6,306,177; 6,319,715; 6,331,578; 6,399,144; and 6,395,029; U.S. patent application Ser. No. 20020022264; and PCT Application Nos. WO 95/24929 and WO 97/47254

What is claimed:

1. A method preparing a biomimetic scaffold comprising:
providing two or more bio-ink solutions; and
co-depositing said bio-ink solutions;
to create said biomimetic scaffold structure.

2. A method preparing a biomimetic scaffold comprising:
providing two or more bio-ink solutions; and
depositing said bio-ink solutions to provide a patterned 3-D concentration gradient of said bio-inks.

3. The method of claim 1, wherein said biomimetic scaffold structure has a 3-D concentration gradient of said bio-ink solutions.

4. The method of any one of claims 1 or 2, wherein said biomimetic scaffold structure has a spatial and temporal concentration gradient of said bio-ink solutions.

5. The method of any one of claims 1 or 2, wherein said bio-ink solidifies, gels, or polymerizes upon deposition.

6. The method of claim 5, wherein said bio-ink solidifies, gels, or polymerizes upon a change in the micro-environment.

7. The method of claim 5, wherein said bio-ink solidifies, gels, or polymerizes upon a change in temperature.

8. The method of claim 5, wherein said bio-ink solidifies, gels, or polymerizes upon a change in pH.

9. The method of claim 5, wherein said bio-ink solidifies, gels, or polymerizes at body-temperature.

10. The method of claim 5, wherein said bio-ink solidifies, gels, or polymerizes at body-temperature.

11. The method of claim 5, wherein said bio-ink solidifies, gels, or polymerizes upon a change in ionic concentration.

12. The method any one of claims 1 or 2, wherein said biomimetic scaffold structure is prepared using a solid freeform fabrication system.

13. The method of claim 12, wherein said solid freeform fabrication system uses a focused micro-dispensing device.

14. The method any one of claims 1 or 2, wherein said bio-inks are co-deposited in situ.

15. The method any one of claims 1 or 2, wherein said bio-inks are co-deposited in a controllable manner.

16. The method any one of claims 1 or 2, wherein said biomimetic scaffold is biocompatible.

17. The method of any one of claims 1 or 2, wherein said biomimetic scaffold is bioresorbable.

18. The method any one of claims 1 or 2, wherein said biomimetic scaffold is biodegradable.

19. The method any one of claims 1 or 2, wherein at least one of said bio-ink solutions is a structural bio-ink solution.

20. The method of claim 19, wherein said structural bio-ink provides said biomimetic scaffold structure mechanical properties.

21. The method of claim 19, wherein said structural bio-ink provides said biomimetic scaffold structure porosity.

22. The method of claim 19, wherein said structural bio-ink provides said biomimetic scaffold structure increased surface area.

23. The method of claim 19, wherein said structural bio-ink solution comprises a hydrogel solution.

24. The method of claim 19, wherein said structural bio-ink solution comprises fibrinogen.

25. The method of claim 24, wherein said fibrinogen is linked to a growth factor.

26. The method of claim 19, wherein said structural bio-ink solution comprises thrombin.

27. The method of any one of claims 1 or 2, wherein a first bio-ink solution is fibrinogen and a second bio-ink solution is thrombin.

28. The method of claim 19, wherein said structural bio-ink solution comprises chitosan.

29. The method of claim 19, wherein said structural bio-ink solution comprises collagen.

30. The method of claim 19, wherein said structural bio-ink solution comprises alginate.

31. The method of claim 19, wherein said structural bio-ink solution comprises poly(N-isopropylacrylamide).

32. The method of claim 19, wherein said structural bio-ink solution comprises hyaluronate.

33. The method of any one of claims 1 or 2, wherein at least one of said bio-ink solutions is a functional bio-ink solution.

34. The method of claim 33, wherein said functional bio-ink provides cell-adhesion properties.

35. The method of claim 33, wherein said functional bio-ink modulates cross-linking within the biomimetic scaffold structure.

36. The method of claim 33, wherein said functional bio-ink modulates the ionic concentration of said biomimetic scaffold structure.

37. The method of claim 33, wherein said functional bio-ink modulates the pH of said biomimetic scaffold structure.

38. The method of claim 33, wherein said functional bio-ink modulates cross-linking within the biomimetic scaffold structure.

39. The method of claim 38, wherein said functional bio-ink comprises a cross-linking agent.

40. The method of claim 39, wherein said cross-linking agent is biocompatible.

41. The method of claim 40, wherein said cross-linking agent is a synthetic cross-linking agent.

42. The method of claim 33, wherein said functional bio-ink comprises a buffer solution.

43. The method of claim 33, wherein said functional bio-ink comprises transglutaminase.

44. The method of any one of claims 1 or 2, wherein at least one of said bio-ink solutions is a therapeutic bio-ink solution.

45. The method of claim 44, wherein said therapeutic bio-ink modulates the immune response.

46. The method of claim 44, wherein said therapeutic bio-ink promotes wound healing.

47. The method of claim 44, wherein said therapeutic bio-ink promotes tissue regeneration.

48. The method of claim 44, wherein said therapeutic bio-ink promotes cell proliferation.

49. The method of claim 44, wherein said therapeutic bio-ink promotes cell differentiation.

50. The method of claim 44, wherein said therapeutic bio-ink promotes angiogenesis.

51. The method of claim 44, wherein said therapeutic bio-ink promotes vessel permeabilization.

52. The method of claim 44, wherein said therapeutic bio-ink comprises agents that elicit a cellular response.

53. The method of claim 52, wherein said agent is selected from the group consisting of growth factors, cytokines, and hormones.

54. The method of claim 53, wherein said agent is a human fibroblast growth factor.

55. The method of claim 53, wherein said agent is a vascular endothelial growth factor.

56. The method of claim 53, wherein said agent is a platelet derived growth factor.

57. The method of claim 53, wherein said agent is an insulin-like growth factor.

58. The method of claim 53, wherein said agent is a human fibroblast growth factor.

59. The method of claim 53, wherein said agent is a bone morphogenic protein.

60. The method of claim 44, wherein said therapeutic bio-ink comprises neurotrophic factors.

61. The method of claim 44, wherein said therapeutic bio-ink comprises small molecules.

62. The method of claim 44, wherein said therapeutic bio-ink comprises signaling molecules

63. The method of claim 44, wherein said therapeutic bio-ink comprises antibodies.

64. The method of claim 44, wherein said therapeutic bio-ink comprises tissue precursor cells.

65. The method of claim 64, wherein said tissue precursor cell is a totipotent stem cell.

66. The method of claim 64, wherein said tissue precursor cell is an embryonic stem cells.

67. The method of claim 64, wherein said tissue precursor cells is selected from the group consisting of osteoblasts, chondrocytes, fibroblasts, and myoblasts.

68. The method of claim 44, wherein said therapeutic bio-ink comprises a nucleic acid.

69. The method of claim 68, wherein said nucleic acid is associated with one or more of the following: nanocaps, colloidal gold, nanoparticulate synthetic particles, and liposomes.

70. A biomimetic scaffold structure prepared by the method of any one of claims 1 or 2, wherein said biomimetic scaffold structure is implantable.

71. A biomimetic scaffold structure of claim 70, wherein said implant is permanent.

72. A biomimetic scaffold structure of claim 70, wherein said implant is biodegradable.

73. A biomimetic scaffold structure prepared by the method of any one of claims 1 or 2, wherein said biomimetic scaffold structure is a skin graft.

74. A biomimetic scaffold structure prepared by the method of any one of claims 1 or 2, wherein said biomimetic scaffold structure is a bioresorbable film.

75. A biomimetic scaffold comprising a 3-D matrix, which matrix has a patterned 3-D concentration gradient of therapeutic bio-inks.

76. An apparatus for dispensing bio-inks onto a surface, the apparatus comprising:
a first micro-dispensing device fluidly connected to a source of a first bio-ink and configured to dispense a volume of the first bio-ink; and
a second micro-dispensing device fluidly connected to a source of a second bio-ink and configured to dispense a volume of the second bio-ink.

77. The apparatus of claim 76, further comprising a movable stage supporting the first micro-dispensing device and the second micro-dispensing device, the movable stage being configured to move the first micro-dispensing device and the second dispensing device relative to the surface.

78. The apparatus of claim 77, wherein the first micro-dispensing device and the second micro-dispensing device are focused to a focal point such that a dispensed volume of the first bio-ink converges with a dispensed volume of the second bio-ink at the focal point, wherein the first micro-dispensing device and the second micro-dispensing device may selectively dispense a focused volume of the first bio-ink and second bio-ink at a plurality of dispensing locations on the surface.

79. The apparatus of claim 76, further comprising a third micro-dispensing device coupled to a source of a third bio-ink and configured to dispense a volume of the third bio-ink.

80. The apparatus of claim 79, further comprising a fourth micro-dispensing device coupled to a source of a fourth bio-ink and configured to dispense a volume of the fourth bio-ink.

81. The apparatus of claim 80, further comprising a fifth micro-dispensing device coupled to a source of a fifth bio-ink and configured to dispense a volume of the fifth bio-ink.

82. The apparatus of claim 81, wherein the first bio-ink, the second bio-ink, the third bio-ink, the fourth bio-ink, and the fifth bio-ink are different compositions.

83. The apparatus of claim 76, further comprising a control system coupled to the first micro-dispensing device and to the second micro-dispensing device, the control system configured to control the volume of first bio-ink and the volume of second bio-ink dispensed.

84. The apparatus of claim 76, wherein at least one of the first micro-dispensing device and the second micro-dispensing device is an ink jet print head.

85. The apparatus of claim 76, wherein at least one of the first micro-dispensing device and the second micro-dispensing device is a micro-dispensing solenoid valve.

86. The apparatus of claim 76, wherein at least one of the first micro-dispensing device and the second micro-dispensing device is a syringe pump.

87. The apparatus of claim 76, wherein at least one of the first micro-dispensing device and the second micro-dispensing device includes a heating unit.

88. The apparatus of claim 76, further comprising a heat source for heating at least a portion of the surface.

88. The apparatus of claim 76, wherein the heat source is an infrared heat source configured to direct infrared light onto at least a portion of the surface.

90. The apparatus of claim 76, wherein at least one of the first micro-dispensing device and the second micro-dispensing device includes a cooling unit.

91. The apparatus of claim 76, further comprising a movable stage supporting the surface and being configured to move the surface relative to the first micro-dispensing device and the second dispensing device.

92. The apparatus of claim 76, wherein at least one of the first bio-ink and the second bio-ink is a structural bio-ink solution.

93. The apparatus of claim 76, wherein at least one of the first bio-ink and the second bio-ink is a functional bio-ink solution.

94. The apparatus of claim 76, wherein at least one of the first bio-ink and the second bio-ink is a therapeutic bio-ink solution.

95. An apparatus for fabricating a biomimetic fibrin scaffold on a surface, the apparatus comprising:
a first micro-dispensing device fluidly connected to a source fibrinogen and configured to dispense a volume of fibrinogen; and
a second micro-dispensing device fluidly connected to a source of thrombin and configured to dispense a volume of thrombin.

96. The apparatus of claim 95, further comprising a movable stage supporting the first micro-dispensing device and the second micro-dispensing device and being configured to move the first micro-dispensing device and the second dispensing device relative to the surface.

97. The apparatus of claim 96, wherein the first micro-dispensing device and the second micro-dispensing device are focused to a focal point such that a dispensed volume of the fibrinogen converges with a dispensed volume of thrombin at the focal point, wherein moving the first micro-dispensing device and the second micro-dispensing device relative to the surface and selectively dispensing a focused volume of fibrinogen and thrombin at a plurality of dispensing locations on the surface creates a biomimetic fibrin scaffold on the surface.

98. An apparatus for in situ dispensing of a bio-ink on a subject, the apparatus comprising:
a first micro-dispensing device fluidly connected to a source of a first bio-ink and configured to dispense a volume of the first bio-ink;
a second micro-dispensing device fluidly connected to a source of a second bio-ink and configured to dispense a volume of the second bio-ink; and
a movable stage supporting the first micro-dispensing device and the second micro-dispensing device and being configured to be connected to a subject, the movable stage being configured to move the first micro-dispensing device and the second micro-dispensing device relative to the subject.

99. The apparatus of claim 98, wherein the movable stage is a stereotactic device.

100. The apparatus of claim 99, wherein the stereotactic device is configured to move the first micro-dispensing device and the second micro-dispensing device along an X-axis, a Y-axis, and a Z-axis.

101. The apparatus of claim 98, wherein the first micro-dispensing device and the second micro-dispensing device are focused to a focal point such that a dispensed volume of the first bio-ink converges with a dispensed volume of the second bio-ink at the focal point, wherein the first micro-dispensing device and the second micro-dispensing device may selectively dispense a focused volume of the first bio-ink and second bio-ink at a plurality of dispensing locations on the subject.

102. An apparatus for fabricating a biomimetic scaffold on a surface, the apparatus comprising:
a first micro-dispensing device fluidly connected to a source of first bio-ink and configured to dispense a volume of the first bio-ink;
a second micro-dispensing device fluidly connected to a source of a second bio-ink and being configured to dispense a volume of the second bio-ink; and
a movable stage supporting the first micro-dispensing device and the second micro-dispensing device and being configured to move the first micro-dispensing device and the second dispensing device relative to the surface, the first micro-dispensing device and the second micro-dispensing device being focused to a focal point such that a dispensed volume of the first bio-ink converges with a dispensed volume of the second bio-ink at the focal point, wherein moving the first micro-dispensing device and the second micro-dispensing device relative to the surface and selectively dispensing a focused volume of the first bio-ink and the second bio-ink at a plurality of dispensing locations on the surface to creates a biomimetic scaffold on the surface.

103. The apparatus of claim 102, further comprising a control system coupled to the first micro-dispensing device and to the second micro-dispensing device, the control system configured to control the volume of first bio-ink and the volume of second bio-ink dispensed at each dispensing location on the surface.

104. The apparatus of claim 103, wherein the control system includes an analysis module configured to analyze a 3-D computer generated model of the biomimetic scaffold to determine the composition of the scaffold.

105. The apparatus of claim 104, wherein the analysis module is configured to subdivide the computer generated model into discrete cube units, and determine the composition of each cube unit.

106. The apparatus of claim 104, wherein the analysis module is configured to determine the porosity of each cube unit.

107. The apparatus of claim 105, wherein the control system includes a mixture-planning module configured to determine a volume of first bio-ink and a volume of second bio-ink to be dispensed in each discrete cube unit.

108. The apparatus of claim 107, wherein the mixture-planning module is configured to maintain a total volume of first bio-ink and second bio-ink dispensed in each discrete cube unit at a selected constant volume.

109. The apparatus of claim 107, wherein the control system includes a dispenser control module coupled to the first micro-dispensing device and to the second micro-dispensing device, the dispenser control module configured to provide control signals to the first micro-dispensing device and to the second micro-dispensing device to control the volume of first bio-ink and a volume of second bio-ink to be dispensed in each discrete cube unit based upon the volumes determined by the mixture-planning module.

110. The apparatus of claim 107, wherein the control system includes a stage control module coupled to the moveable stage and configured to control the motion of the first micro-dispensing device and to the second micro-dispensing device.

111. A hand-held instrument comprising:
an instrument frame having a handle sized and shaped to be held by a user;
a first micro-dispensing device coupled to the instrument frame and fluidly connected to a source of a first bio-ink, the first micro-dispensing device being configured to dispense a volume of the first bio-ink; and
a second micro-dispensing device coupled to the instrument frame and fluidly connected to a source of a second bio-ink, the second micro-dispensing device configured to dispense a volume of the second bio-ink.

112. The hand held instrument of claim 111, wherein the first micro-dispensing device and the second micro-dispensing device are focused to a focal point such that a dispensed volume of the first bio-ink converges with a dispensed volume of the second bio-ink at the focal point.

113. The hand held instrument of claim 111, wherein the instrument frame further comprises
a first reservoir containing the source of first bio-ink, and
a second reservoir containing the source of second bio-ink.

114. A hand-held instrument comprising:
an instrument frame having a handle sized and shaped to be held by a user;
a first micro-dispensing device coupled to the instrument frame and fluidly connected to a source of a fibrinogen, the first micro-dispensing device being configured to dispense a volume of the fibrinogen; and
a second micro-dispensing device coupled to the instrument frame and fluidly connected to a source of a thrombin, the second micro-dispensing device configured to dispense a volume of the thrombin, the first micro-dispensing device and the second micro-dispensing device being focused to a focal point such that a dispensed volume of the fibrinogen converges with a dispensed volume of thrombin at the focal point.

115. In a minimally invasive surgical instrument, an apparatus for dispensing a bio-ink in vivo comprising:
a first micro-dispensing device coupled to the instrument and fluidly connected to a source of a bio-ink, the first micro-dispensing device being configured to dispense a volume of the bi-ink onto a surface of a subject.
